(19) United States
(12) Patent Application Publication
(10) Pub. No.: US 2026/0345976 A1
(43) Pub. Date: Nov. 5, 2026

(54) NASAL DILATING DEVICE

(71) Applicant: Fissiontech LLC, New York City, NY (US)

(72) Inventors: Xiangyun KONG, Philadelphia, PA (US); Christopher John BATON, Dover Plains, NY (US)

(21) Appl. No.: 19/641,645

(22) Filed: Apr. 8, 2026

Related U.S. Application Data
(63) Continuation-in-part of application No. 19/295,482, filed on Aug. 8, 2025, now Pat. No. 12,616,597.
(60) Provisional application No. 63/797,798, filed on Apr. 30, 2025.

(51) Int. Cl. A61M 29/00; A61F 5/56

(52) U.S. Cl. CPC A61M 29/00; A61F 5/56; A61M 2210/0618

(57) ABSTRACT

Included in the present disclosure is a device, including a bridge having a first end and a second end opposite the first end. In some embodiments, the device includes a first arm detachably coupled to the first end. According to some embodiments, the device includes a second arm detachably coupled to the second end. The device may be configured to detachably couple with a nose of a user.

### CROSS-REFERENCE TO RELATED APPLICATIONS

The entire contents of the following application are incorporated herein: U.S. Nonprovisional Patent Application No. 19/295,482; filed on Aug. 8, 2025; and entitled NASAL DILATING DEVICE.

The entire contents of the following application are incorporated herein: U.S. Provisional Patent Application No. 63/797,798; filed on Apr. 30, 2025; and entitled NASAL DILATING DEVICE.

### FIELD OF TECHNOLOGY

The present disclosure relates to devices for opening nasal airways.

### BACKGROUND

Nasal dilators help to keep a person’s nasal passages open in order to facilitate their breathing. This may include times when deeper breaths are taken, such as during sleep or exercise. Many nasal dilators operate by physically widening the nostrils or the nasal valve area, which is the narrowest part of the nasal airway.

Nasal dilators are generally used in order to reduce snoring, improve airflow during sleep, helping with problems such as mild sleep apnea or nasal congestion, and to help with athletic performance through easing nasal breathing.

### SUMMARY

Included in the present disclosure is a device, including a bridge having a first end and a second end opposite the first end. In some embodiments, the device includes a first arm detachably coupled to the first end. According to some embodiments, the device includes a second arm detachably coupled to the second end. The device may be configured to detachably couple with a nose of a user.

In some embodiments, the first end of the bridge includes a first insert. According to some embodiments, the second end of the bridge comprises a second insert. The first insert may include a first female coupling mechanism. In some embodiments, the second insert includes a second female coupling mechanism.

According to some embodiments, the first end includes a first female coupling mechanism. The second end may include a second female coupling mechanism. In some embodiments, the bridge is configured to change in width by about five millimeters when in use. According to some embodiments, the bridge includes a spring constant configured to apply pressure to nostrils of the nose to thereby open a nasal airway. The bridge may include i) stainless steel, ii) high-precision phosphorus copper, iii) titanium, iv) polymer, v) polycarbonate, vi) steel, or vii) combinations thereof.

In some embodiments, the first arm includes a first slot. According to some embodiments, the second arm comprises a second slot. The first slot may be configured to detachably couple with the first insert. In some embodiments, the second slot is configured to detachably couple with the second insert. According to some embodiments, the first slot is configured to detachably couple with the first end. The second slot may be configured to detachably couple with the second end.

In some embodiments, the device further includes a first male coupling mechanism disposed within the first slot. According to some embodiments, the device further includes a second male coupling mechanism disposed within the second slot. The first male coupling mechanism may be configured to detachably couple with the first female coupling mechanism when the first slot is coupled with the first insert or first end. In some embodiments, the second male coupling mechanism is configured to detachably couple with the second female coupling mechanism when the second slot is coupled with the second insert or second end. According to some embodiments, the first arm and the second arm each include i) silicon, ii) plastic, iii) polymer, or iv) combinations thereof.

The first arm may include a first nasal coupling mechanism. In some embodiments, the second arm includes a second nasal coupling mechanism. According to some embodiments, the first nasal coupling mechanism is configured to detachably couple the first arm to a first ala of the nose. The second nasal coupling mechanism may be configured to detachably couple the second arm to a second ala of the nose. In some embodiments, the device further includes a first pair of magnets disposed in the first nasal coupling mechanism, the first pair of magnets configured to detachably couple the first arm to the first ala. According to some embodiments, the device includes a second pair of magnets disposed in the second nasal coupling mechanism, the second pair of magnets configured to detachably couple the second arm to the second ala.

The device may be configured to apply pressure to nostrils of the nose, thereby opening a nasal airway. In some embodiments, the bridge includes a bridge position indicator, the first arm includes a first arm position indicator, and the second arm includes a second arm position indicator. According to some embodiments, the bridge position indicator and the first arm position indicator are configured to align the bridge with the first arm. The bridge position indicator and the second arm position indicator may be configured to align the bridge with the second arm. In some embodiments, i) the bridge position indicator, ii) the first arm position indicator, iii) the second arm position indicator, or iv) combinations thereof are triangular.

According to some embodiments, the arm includes an arm size indicator. The bridge includes a bridge size indicator. A height of the bridge may be from 6.667 millimeters (mm) to 9.6 mm. In some embodiments, a width of the bridge, less the first insert and the second insert, is from 20.458 mm to 35.112 mm. According to some embodiments, a width of i) the first insert, ii) the second insert, or iii) both is from 4.167 mm to 6 mm. A height of i) the first insert, ii) the second insert, or iii) both may be from 4.583 mm to 6.6 mm.

In some embodiments, a depth of the arm is from 9.008 mm to 13.524 mm. According to some embodiments, a height of an inner portion of the arm is from 12.983 mm to 20.592 mm. A width of an ala contacting portion of the arm may be from 6.75 mm to 9.72 mm. In some embodiments, an angle of travel of the bridge is from 80 degrees to 144 degrees.

Also included in the present disclosure is a system including a device as described above. In some embodiments, the system includes a case including an interior, the case sized and configured to hold the device within the interior.

According to some embodiments, the case includes i) plastic, ii) foam, iii) faux fur, iv) nylon, or v) combinations thereof. The plastic may include i) transparent plastic, ii) translucent plastic, iii) opaque plastic, iv) colored plastic, or v) combinations thereof.

In some embodiments, a height of the case is from 20.758 mm to 29.892 mm. According to some embodiments, a length of the case is from 59.608 mm to 85.836 mm. A width of the case may be from 34.675 mm to 49.932 mm.

Also included in the present disclosure is a kit, including a first device as described above, the first device including a first bridge, a first arm, and a second arm. The kit may include a second device as described above, the second device including a second bridge, a third arm, and a fourth arm.

In some embodiments, the first bridge includes a first width. According to some embodiments, the second bridge includes a second width. The first width may be different than the second width.

In some embodiments, the first bridge includes a first material. According to some embodiments, the second bridge includes a second material. The first material may be different than the second material.

In some embodiments, the first bridge includes a first spring constant. According to some embodiments, the second bridge includes a second spring constant. The first spring constant may be different than the second spring constant.

In some embodiments, the first arm includes a first nasal coupling mechanism defining a first width. According to some embodiments, the second arm includes a second nasal coupling mechanism defining a second width. The third arm may include a third nasal coupling mechanism defining a third width. In some embodiments, the fourth arm includes a fourth nasal coupling mechanism defining a fourth width. According to some embodiments, the first width and the second width are the same. The third width and the fourth width may be the same. In some embodiments, the first width and the second width are different from the third width and the fourth width.

According to some embodiments, the first arm includes a first nasal coupling mechanism configured to be inserted into a nostril a first depth. The second arm may include a second nasal coupling mechanism configured to be inserted into a nostril a second depth. In some embodiments, the third arm includes a third nasal coupling mechanism configured to be inserted into a nostril a third depth. According to some embodiments, the fourth arm includes a fourth nasal coupling mechanism configured to be inserted into a nostril a fourth depth. The first depth and the second depth may be the same. In some embodiments, the third depth and the fourth depth are the same. According to some embodiments, the first depth and the second depth are different from the third depth and the fourth depth.

The first arm may include a first material. In some embodiments, the second arm includes a second material, the third arm includes a third material, and the fourth arm includes a fourth material. According to some embodiments, the first material and the second material are the same, The third material and the fourth material may be the same. In some embodiments, the first material and the second material are different from the third material and the fourth material.

According to some embodiments, the kit further includes a case including an interior, the case sized and configured to hold the first device and the second device within the interior. The case may include i) plastic, ii) foam, iii) faux fur, iv) nylon, or v) combinations thereof. In some embodiments, the plastic includes i) transparent plastic, ii) translucent plastic, iii) opaque plastic, iv) colored plastic, or v) combinations thereof.

According to some embodiments, a height of the case is from 20.758 mm to 29.892 mm. A length of the case may be from 59.608 mm to 85.836 mm. In some embodiments, a width of the case is from 34.675 mm to 49.932 mm.

The disclosure further includes a device including a first handle portion and a second handle portion. In some embodiments, the first handle portion is configured to receive a first end of a bridge configured to be placed about the bridge of a nose of a user, and the second handle portion is configured to receive a second end of the bridge, wherein the second end of the bridge is located opposite the first end of the bridge. When the bridge is coupled to the first handle portion and the second handle portion, the first handle portion and the second handle portion may be used to alter a radius of curvature of the bridge.

In some embodiments, a first end of the first handle portion includes a first slot configured to receive the first end of the bridge, and a first end of the second handle portion includes a second slot configured to receive the second end of the bridge. The first end of the bridge may include a first insert having a first female coupling mechanism, and the second end of the bridge may include a second insert having a second female coupling mechanism. In some embodiments, the first slot is configured to receive the first insert, and the second slot is configured to receive the second insert.

The device may further include a first male coupling mechanism disposed within the first slot and a second male coupling mechanism disposed within the second slot. In some embodiments, the first male coupling mechanism is configured to detachably couple with the first female coupling mechanism when the first slot is coupled with the first insert. The second male coupling mechanism may be configured to detachably couple with the second female coupling mechanism when the second slot is coupled with the second insert.

In some embodiments, the first male coupling mechanism includes at least one protrusion located along a wall within the first slot, and the first female coupling mechanism includes at least one aperture configured to at least partially receive the at least one protrusion of the first male coupling mechanism. The second male coupling mechanism may include at least one protrusion located along a wall within the second slot, and the second female coupling mechanism may include at least one aperture configured to at least partially receive the at least one protrusion of the second male coupling mechanism.

According to some embodiments, the first slot includes a first side portion defining a first depth, a second side portion spaced from the first side portion and defining the first depth, and a middle portion located between the first side portion and the second side portion, wherein the middle portion defines a second depth that is greater than the first depth. The second slot may include a first side portion defining the first depth, a second side portion spaced from the first side portion and defining the first depth, and a middle portion located between the first side portion and the second side portion, wherein the middle portion defines the second depth. In some embodiments, the first insert defines a first length configured to correspond to the second depth of the middle portion of the first slot, and the second insert defines a second length configured to correspond to the second depth of the middle portion of the second slot.

The first handle portion may include a first face and a second face located opposite the first face of the first handle portion. In some embodiments, each of the first face and the second face of the first handle portion includes at least one grip area. The second handle portion may include a first face and a second face located opposite the first face of the second handle portion. In some embodiments, each of the first face and the second face of the second handle portion includes at least one grip area.

The disclosure further includes a kit, including a tool including a first handle portion and a second handle portion, and a bridge including a first end and a second end located opposite the first end, wherein the bridge is configured to be placed about a bridge of a nose of a user. According to some embodiments, the first handle portion is configured to receive at least a portion of the first end of the bridge, and the second handle portion is configured to receive at least a portion of the second end of the bridge. When the bridge is coupled to the first handle portion and the second handle portion, the first handle portion and the second handle portion may be used to alter a radius of curvature of the bridge.

In some embodiments, the first handle portion includes a first slot and a first male coupling mechanism disposed within the first slot. The second handle portion may include a second slot and a second male coupling mechanism disposed within the second slot. In some embodiments, the first end of the bridge includes a first insert having a first female coupling mechanism, and the second end of the bridge includes a second insert having a second female coupling mechanism. The first slot may be configured to receive the first insert, and the second slot may be configured to receive the second insert.

According to some embodiments, the first male coupling mechanism is configured to detachably couple with the first female coupling mechanism when the first slot is coupled with the first insert. The second male coupling mechanism may be configured to detachably couple with the second female coupling mechanism when the second slot is coupled with the second insert.

In some embodiments, the kit further includes a first arm configured to detachably couple to the first end of the bridge, wherein at least a portion of the first arm is configured to be inserted within a first nostril of the user. The kit may also include a second arm configured to detachably couple to the second end of the bridge, wherein at least a portion of the second arm is configured to be inserted within a second nostril of the user. In some embodiments, the bridge has a spring constant such that the bridge is configured to independently pull the first arm and the second arm away from one another to thereby open a nasal airway.

The first end of the bridge may include a first insert having a first female coupling mechanism. In some embodiments, the second end of the bridge includes a second insert having a second female coupling mechanism. The first arm may include a first slot and a first male coupling mechanism disposed within the first slot, and the second arm may include a second slot and a second male coupling mechanism disposed within the second slot. According to some embodiments, the first male coupling mechanism is configured to detachably couple with the first female coupling mechanism when the first slot is coupled with the first insert, and the second male coupling mechanism is configured to detachably couple with the second female coupling mechanism when the second slot is coupled with the second insert.

The bridge may include a first bridge defining a first bridge size. In some embodiments, the first arm and the second arm define a first set of arms defining a first arm size. The kit may also include a second bridge defining a second bridge size that is different from the first bridge size, and a third bridge defining a third bridge size that is different from the first bridge size and the second bridge size. In some embodiments, the kit includes a second set of arms defining a second arm size that is different from the first arm size, the second set of arms having a third arm and a fourth arm. The kit may also include a third set of arms defining a third arm size that is different from the first arm size and the second arm size, the third set of arms having a fifth arm and a sixth arm.

In some embodiments, the kit includes a case configured to securably store at least one of the tool, the first bridge, the second bridge, the third bridge, the first set of arms, the second set of arms, and the third set of arms. The tool may be sized and configured to detachably couple to any of the first bridge, the second bridge, and the third bridge.

The foregoing, and other features and advantages of the invention, will be apparent from the following, more particular description of the preferred embodiments of the invention, the accompanying drawings, and the claims.

### BRIEF DESCRIPTION OF DRAWINGS

These and other features, aspects, and advantages are described below with reference to the drawings, which are intended to illustrate, but not to limit, the invention. In the drawings, like characters denote corresponding features consistently throughout similar embodiments.

FIG. 1 illustrates a user using a nasal dilating device, according to some embodiments.

FIG. 2 illustrates a perspective view of a nasal dilating device, according to some embodiments.

FIG. 3A illustrates a front view of the nasal dilating device of FIG. 1, according to some embodiments.

FIG. 3B illustrates a back view of the nasal dilating device of FIG. 1, according to some embodiments.

FIG. 3C illustrates a top view of the nasal dilating device of FIG. 1, according to some embodiments.

FIG. 3D illustrates a bottom view of the nasal dilating device of FIG. 1, according to some embodiments.

FIG. 3E illustrates a side view of the nasal dilating device of FIG. 1, according to some embodiments.

FIG. 4A illustrates a front exploded view of the nasal dilating device of FIG. 1, according to some embodiments.

FIG. 4B illustrates a back exploded view of the nasal dilating device of FIG. 1, according to some embodiments.

FIG. 4C illustrates a top exploded view of the nasal dilating device of FIG. 1, according to some embodiments.

FIG. 5A illustrates a perspective view of a bridge, according to some embodiments.

FIG. 5B illustrates a front view of the bridge of FIG. 5A, according to some embodiments.

FIG. 5C illustrates a back view of the bridge of FIG. 5A, according to some embodiments.

FIG. 5D illustrates a diagrammatic side view of the bridge of FIG. 5A, according to some embodiments.

FIG. 5E illustrates a diagrammatic back view of the bridge of FIG. 5A, according to some embodiments.

FIG. 6A illustrates a perspective view of an arm, according to some embodiments.

FIG. 6B illustrates a side view of the arm of FIG. 6A, according to some embodiments.

FIG. 6C illustrates a front view of the arm of FIG. 6A, according to some embodiments.

FIG. 6D illustrates a back view of the arm of FIG. 6A, according to some embodiments.

FIG. 6E illustrates a diagrammatic front view of the arm of FIG. 6A, according to some embodiments.

FIG. 6F illustrates a diagrammatic side view of the arm of FIG. 6A, according to some embodiments.

FIGS. 7A, 7B, and 7C illustrate partial views of a slot of an arm, according to some embodiments.

FIG. 8 illustrates a side view of an arm, according to some embodiments.

FIG. 9 illustrates a diagrammatic back view of a nasal dilating device being put together, according to some embodiments.

FIG. 10A illustrates a back view of a bridge, according to some embodiments.

FIG. 10B illustrates a side view of an arm, according to some embodiments.

FIG. 11A illustrates a perspective view of a case, according to some embodiments.

FIG. 11B illustrates a perspective view of the case of FIG. 11A in an open configuration, according to some embodiments.

FIG. 11C illustrates a perspective view of the case of FIG. 11A in an open configuration with a nasal opening device inside, according to some embodiments.

FIG. 11D illustrates a diagrammatic front view of the case of FIG. 11A, according to some embodiments.

FIG. 11E illustrates a diagrammatic top view of the case of FIG. 11A, according to some embodiments.

FIG. 12 illustrates a packing tray, according to some embodiments.

FIG. 13 illustrates a perspective view of a tool coupled to the bridge of FIG. 5A, according to some embodiments.

FIG. 14 illustrates an exploded view of the tool and bridge of FIG. 13, according to some embodiments.

FIGS. 15A and 15B illustrate the tool and bridge of FIG. 13, where the bridge is shown with different radii of curvature, according to some embodiments.

FIG. 16 illustrates a perspective view of the tool of FIG. 13, according to some embodiments.

FIG. 17 illustrates a top view of the tool of FIG. 13, according to some embodiments.

FIGS. 18 and 19 illustrate top views of a slot of the tool of FIG. 13, according to some embodiments.

FIG. 20 illustrates a partial X-ray view of the tool of FIG. 13, according to some embodiments.

FIGS. 21A and 21B illustrate perspective views of ends of the bridge of FIG. 5A, according to some embodiments.

FIG. 22 illustrates a front view of the tool of FIG. 13, according to some embodiments.

FIG. 23 illustrates a back view of the tool of FIG. 13, according to some embodiments.

FIG. 24 illustrates a side view of the tool of FIG. 13, according to some embodiments.

FIG. 25 illustrates a back view of the packing tray of FIG. 12, according to some embodiments.

FIG. 26 illustrates a perspective back view of the packing tray of FIG. 12, according to some embodiments.

FIG. 27 illustrates a diagrammatic view of a kit, according to some embodiments.

### DETAILED DESCRIPTION OF THE INVENTION

External nasal dilators physically attach to a person’s nose along an exterior portion of the nose. These external nasal dilators traditionally use magnets or adhesive(s) in order to pull upon the nostrils, thereby opening the nasal airway. However, such devices include multiple shortcomings that the present disclosure serves to resolve.

For example, devices using adhesive are single-use and must be disposed of each time the device is used. Devices using magnets also include adhesive(s) in order to adhere the magnet to a person’s nose. Additionally, in order for the adhesive to be effective, the person must thoroughly wash the location to which they are going to stick the adhesive prior to each use. Finally, these prior devices are “one-size-fits-all” in that there are no customization options available.

Disclosed herein, in some aspects, is a nasal dilating device configured to couple with a user’s nose to dilate one or more nasal airways and/or nostrils. The device of the present disclosure may be modular, including two arms that interact with a person’s nose, as well as a bridge that connects the two arms. Multiple size, depth, and material options may be made available to the user, permitting the user to customize their experience, such as by way of how deep the arms enter the person’s nose, how long the bridge is, how stiff the bridge is, and how soft the arms are. Additionally, because adhesive may not be necessary, the device may be reusable, instead of a one-time-use device. Moreover, there may be no disposable components in the device of the present disclosure, meaning fewer items going into the trash, and fewer purchases needing to be made by the user.

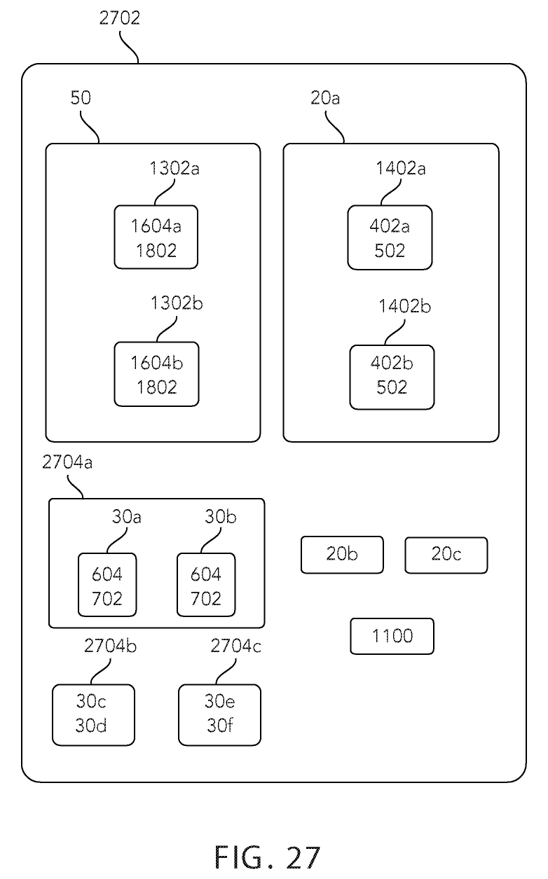

Throughout the present disclosure, reference may be made to a “kit.” This “kit” may include any number of bridges and arms with any variety of features for providing a customizable, modular experience to the user. A diagram of the kit is illustrated in FIG. 27.

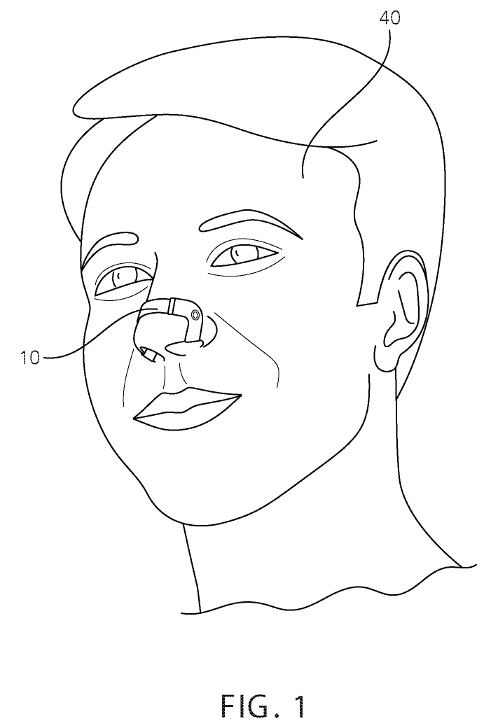

FIG. 1 illustrates a user 40 using a nasal dilating device 10, according to some embodiments. The nasal dilating device 10 may partially insert into one of the user’s 40 nostrils, wrap around the ala of the user’s 40 nose, across/around the dorsum of the user’s 40 nose, wrap around the ala on the opposite side of the user’s 40 nose, and/or partially insert into the other user’s nostril. In application, the entire nasal dilating device 10 may be inserted into both of the user’s 40 nostrils at the same time.

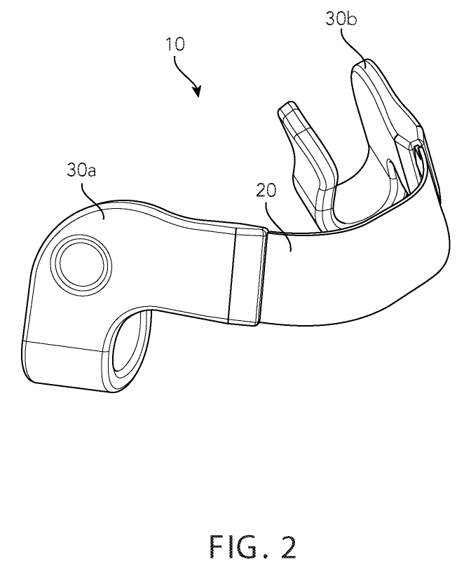

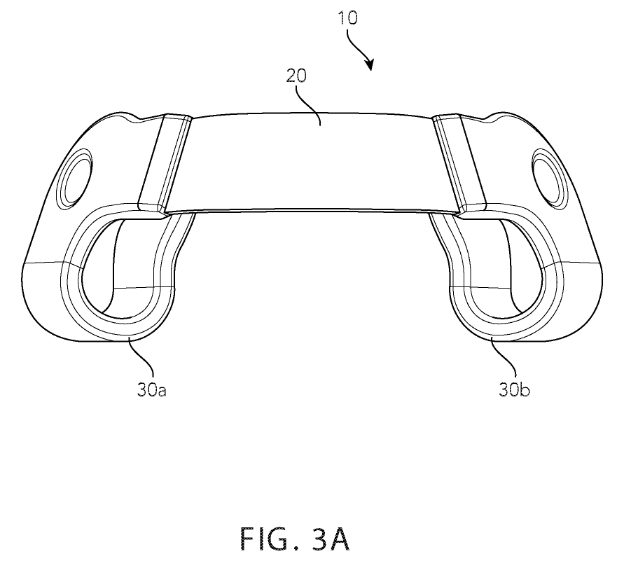

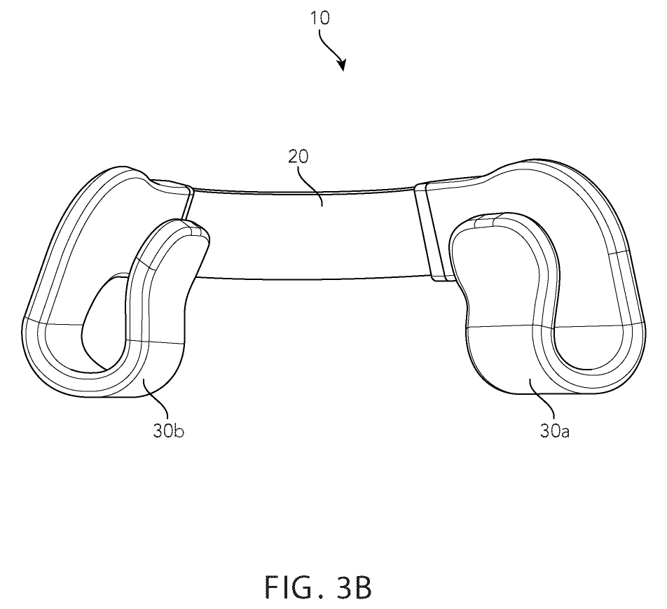

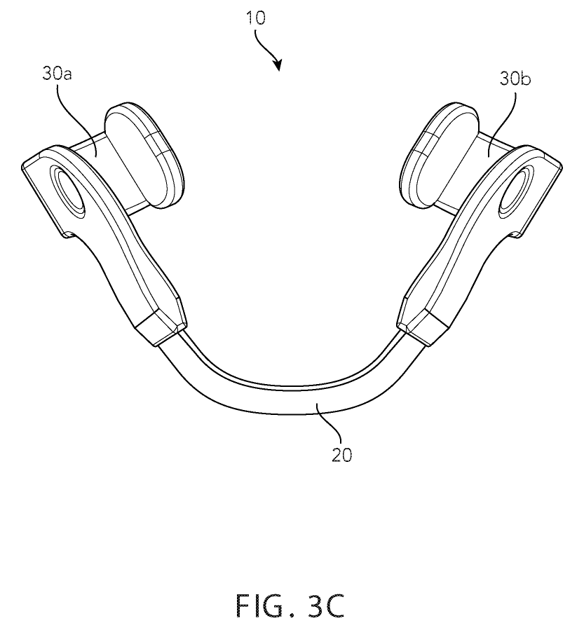

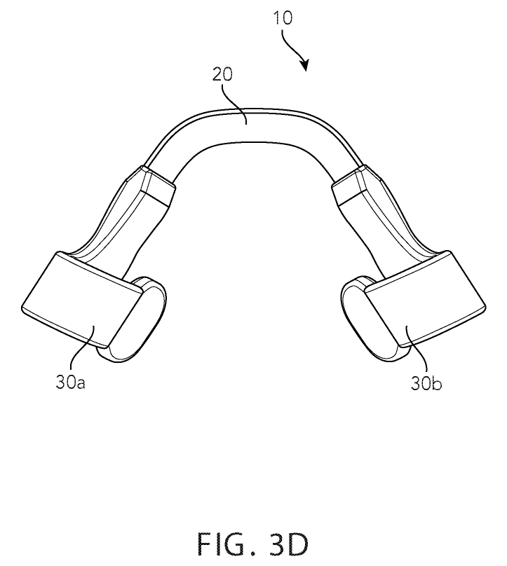

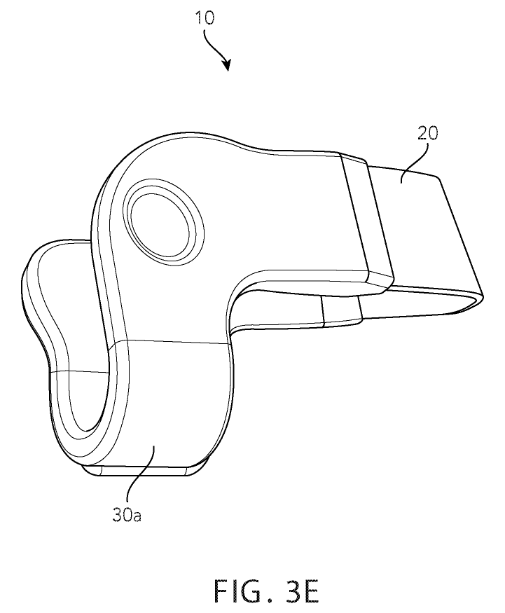

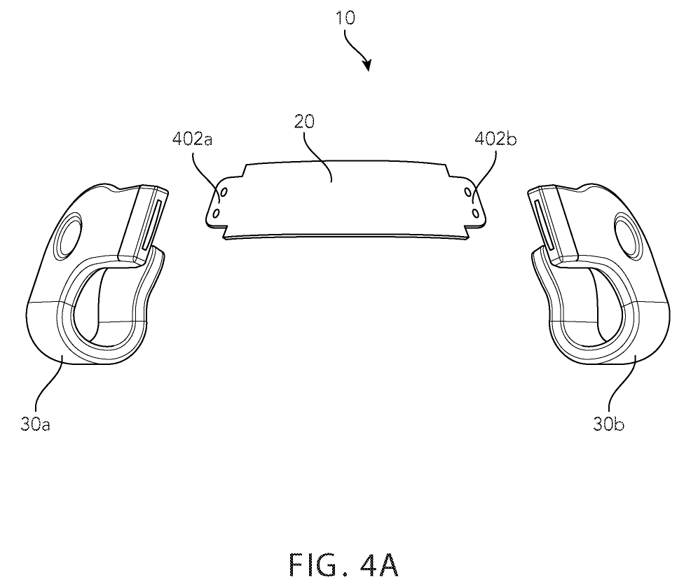

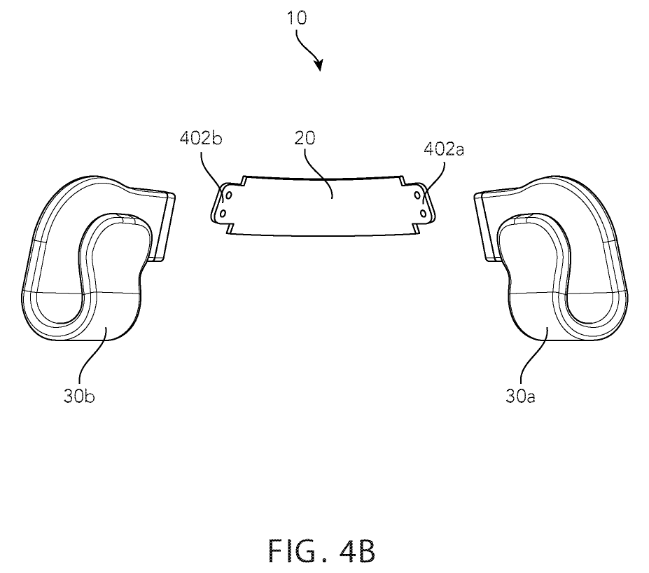

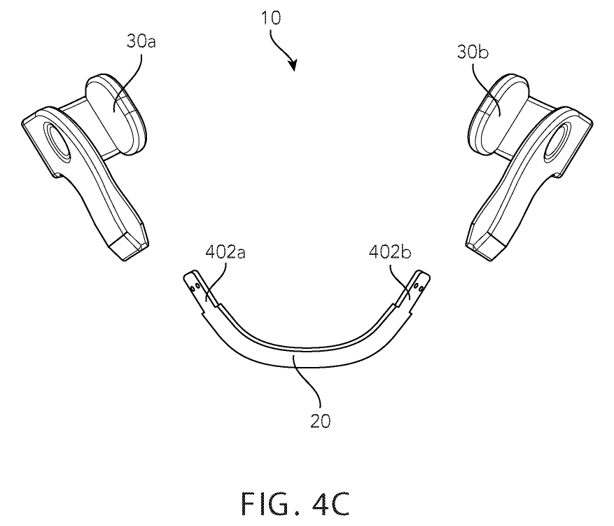

FIG. 2 illustrates a perspective view of a nasal dilating device 10, according to some embodiments. FIG. 3A illustrates a front view, FIG. 3B illustrates a back view, FIG. 3C illustrates a top view, FIG. 3D illustrates a bottom view, FIG. 3E illustrates a side view, FIG. 4A illustrates a front exploded view, FIG. 4B illustrates a back exploded view, and FIG. 4C illustrates a top exploded view of the nasal dilating device 10.

As shown in FIGS. 2-4C, the nasal dilating device 10 includes a bridge 20, a first arm 30a, and a second arm 30b on either side of the bridge 20. The first arm 30a and the second arm 30b may be detachable from the bridge 20. As described below with respect to the specific components, variations of the bridge 20 and the arms 30 may be provided in order to best match up with a nose of a given user onto which the nasal dilating device 10 is being placed. Such modular customization of the nasal dilating device may facilitate comfort for the user.

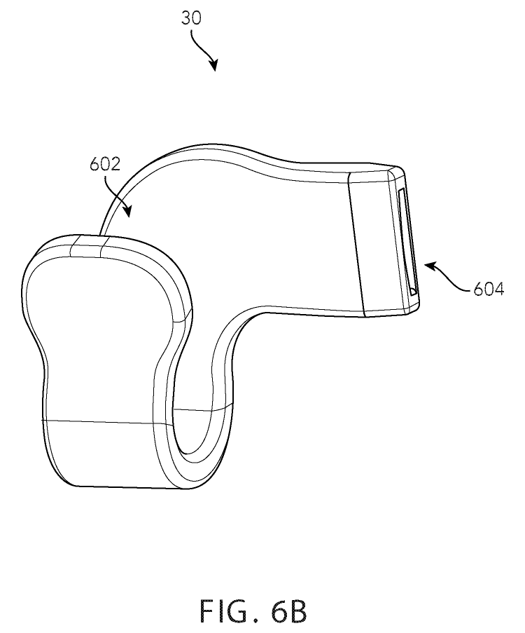

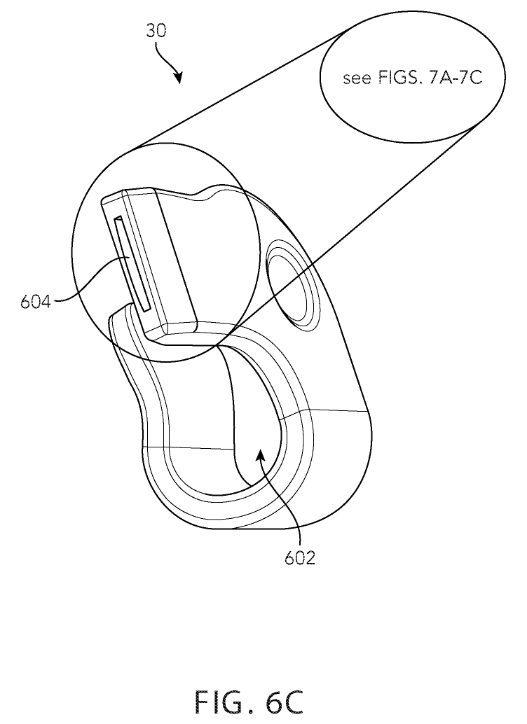

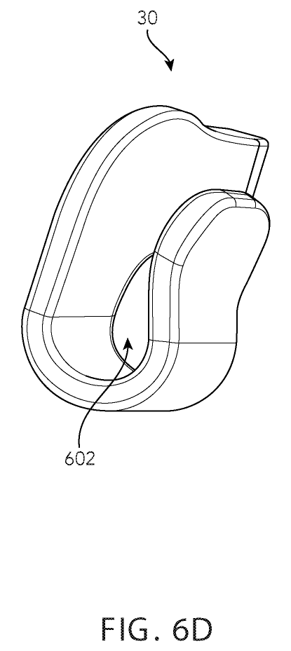

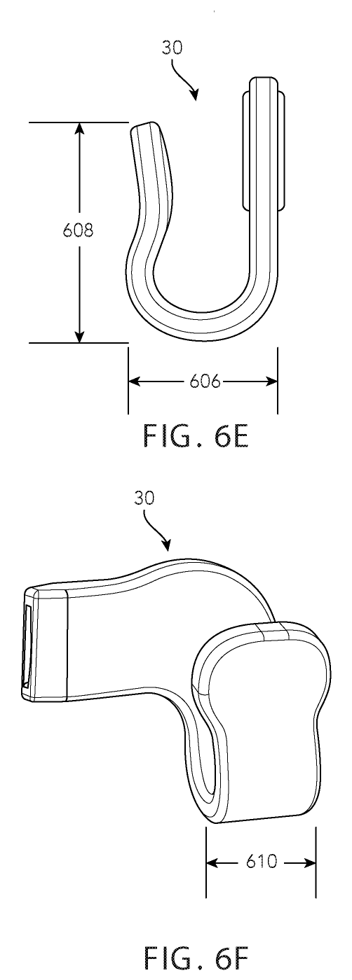

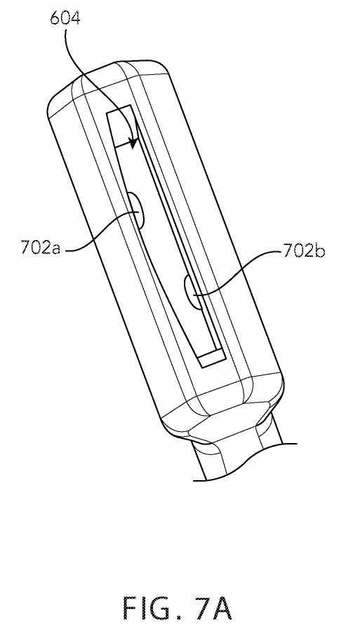

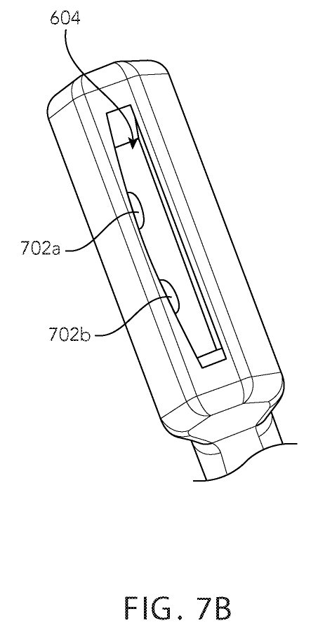

As seen in FIGS. 4A-4C, the bridge 20 may include inserts 402 for coupling to slots in arms 30 (not labeled here, but see e.g., 604 in FIGS. 6A-6D). These inserts 402 may be of a height and length which interact with the slots of the arms 30, such as to make a strong coupling, while not detracting from the ability to remove the arms 30 from the bridge 20. As also shown in FIGS. 4A-4C, the bridge includes an insert 402a on one side of the bridge 20, and an insert 402b on a side of the bridge 20 opposite the insert 402a. Insert 402a may align and/or be configured to couple to arm 30a, and insert 402b may align and/or be configured to couple to arm 30b.

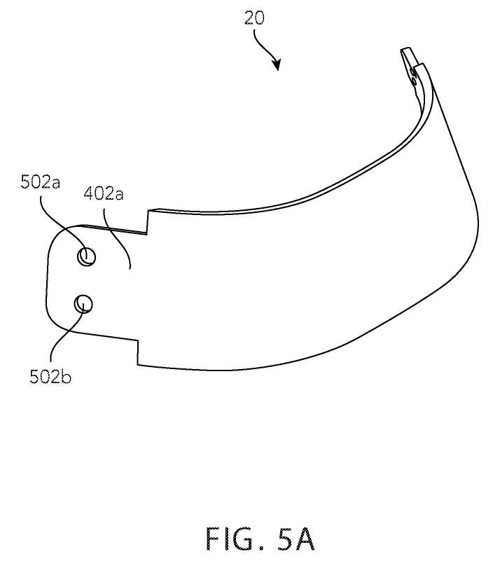

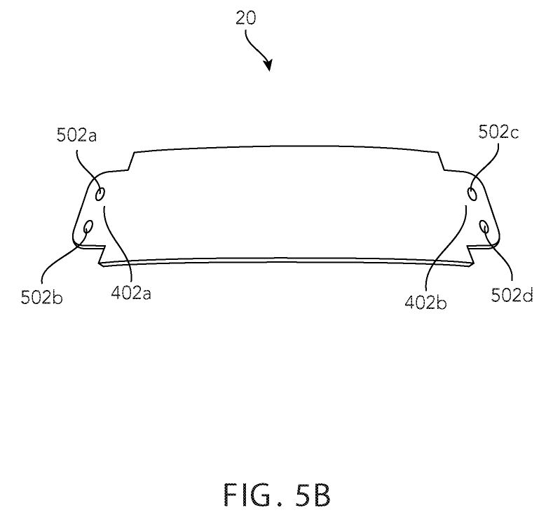

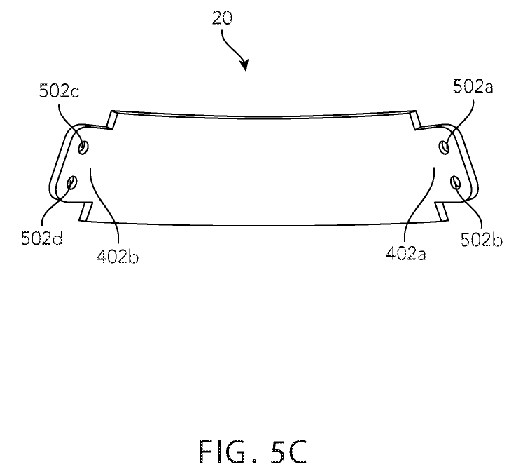

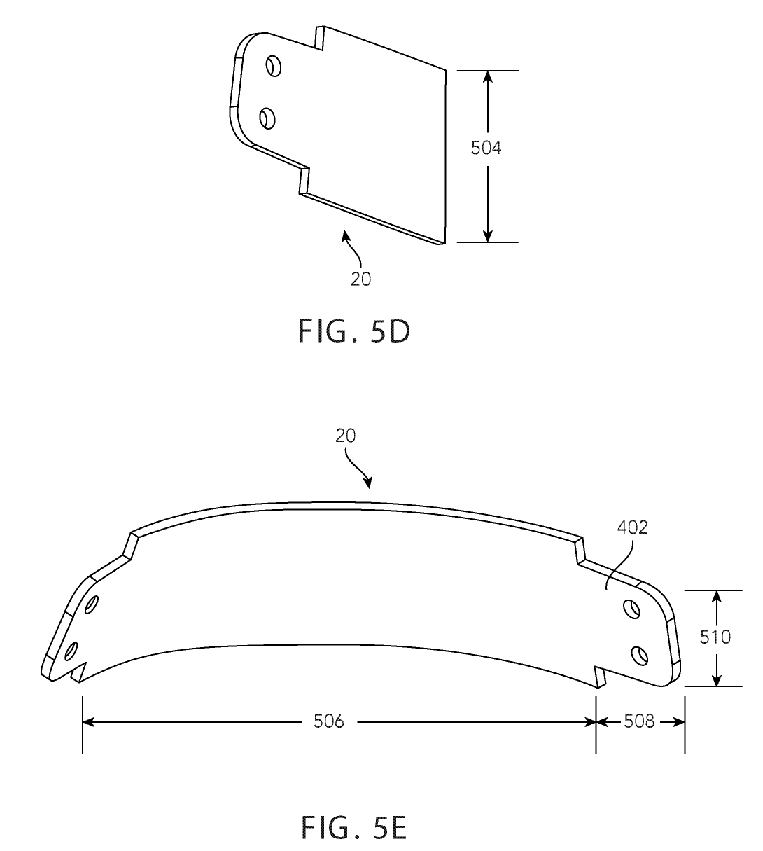

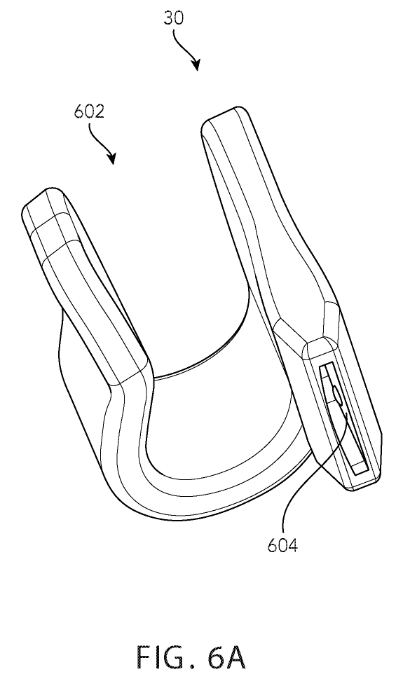

FIG. 5A illustrates a perspective view of a bridge 20, according to some embodiments. FIG. 5B illustrates a front view and FIG. 5C illustrates a back view thereof. FIG. 5D illustrates a diagrammatic side view, and FIG. 5E illustrates a diagrammatic back view thereof. As shown in FIGS. 5A-5C, the bridge 20 may include female coupling mechanisms 502 disposed about the insert 402. The female coupling mechanisms 502 may be holes or recessed portions disposed within the insert 402.

As shown in FIGS. 5A-5C, two female coupling mechanisms 502a and 502b are disposed within insert 402a, and two female coupling mechanisms 502c and 502d are disposed within insert 402b. In alternate embodiments, one female coupling mechanism 502 is disposed within each insert 402. In further embodiments, a greater number than two female coupling mechanisms 502 are disposed within each insert 402. In still further embodiments, the number of female coupling mechanisms 502 disposed within insert 402a does not match the number of female coupling mechanisms 502 disposed within insert 402b.

The bridge 20 may include a height and a width. The height and width of the bridge 20 may be sized and/or configured to facilitate interaction with a user’s dorsum (i.e., the bridge of a user’s nose). In embodiments including a kit, multiple bridges 20 may be included, the bridges 20 having different heights and/or widths from one another. In this way, the user may select a bridge 20 having a height and a width that match or at least substantially match the size and/or shape of the user’s nose. In some embodiments, the bridge experiences about five millimeters in length change while in use. According to some embodiments, the bridge includes a spring constant configured to apply pressure to the nostrils in order to open the nasal airway. In embodiments including a kit, different bridges may include different changes in length during use, and/or different spring constants. For example dimensions of the bridge 20, refer to FIGS. 5D and 5E below.

The device may be used by compressing (e.g., bending) the bridge, at least partially, such that the arms may be inserted about a user’s alae. Once the arms are secured to the user’s alae, the tension in the bridge due to the bridge's inherent spring constant will attempt to bring the bridge back to its original position, outside of external force. This tension in the bridge will cause the arms to separate the alae, along with further portions of the nostril, away from the septum, thereby opening the nasal cavity. This, in turn, may facilitate the opening of the nasal airway, easing air passage through this nasal airway.

The bridge may be formed from stainless steel, high-precision phosphorus copper, titanium, polymer, polycarbonate, and/or steel. In embodiments including a kit, multiple bridges 20 may be included, the bridges 20 being made from different materials from one another. In this way, the user may select a bridge 20 based on a preferred material, such as to avoid an allergy, or discomfort caused by some materials to the specific user.

FIG. 5D illustrates a diagrammatic side view of a portion of the bridge 20 of FIG. 5A, and FIG. 5E illustrates a diagrammatic back view thereof. A height 504 of the bridge 20 is shown in FIG. 5D, and a width 506 of the bridge 20 (less the width of the inserts on either side of the bridge 20), a width 508 of the insert 402, and a height 510 of the insert 402 are shown in FIG. 5E.

The height 504 of the bridge 20 may be from 6.667 millimeters (mm) to 9.6 mm. The width 506 of the bridge 20, less the inserts, may be from 20.458 mm to 35.112 mm. The width 508 of the insert 402 may be from 4.167 mm to 6 mm. The height 510 of the insert 402 may be from 4.583 mm to 6.6 mm. As disclosed above, different bridges 20 may be present in a kit, and the different bridges 20 may each have different dimensions with respect to the height 504 of the bridge 20, the width 506 of the bridge 20 less the inserts, the width 508 of the inserts 402, and/or the height 510 of the inserts 402.

Additionally, the bridge 20 may have an angle of travel between the inserts 402. This angle of travel may be from 80 degrees to 144 degrees. Stated another way, the angle of travel may be from 1.396 radians to 2.513 radians.

FIG. 6A illustrates a perspective view of an arm 30, according to some embodiments. FIG. 6B illustrates a side view, FIG. 6C illustrates a front view, and FIG. 6D illustrates a back view thereof. FIG. 6E illustrates a diagrammatic front view, and FIG. 6F illustrates a diagrammatic side view thereof. For illustration purposes, only one arm 30 is shown, as the opposing arm 30 may be identical, with the features mirrored. For the purposes of FIGS. 6A-6D, arm 30b from FIGS. 1-3C is illustrated and referred to here as arm 30.

As shown in FIGS. 6A-6D, the arm 30 may include a nasal coupling mechanism 602 and a slot 604. As described above with respect to FIGS. 4A-4C, the slot 604 may be configured to detachably couple with the inserts 402 on the bridge 20. For example, the slot 604 may be configured to receive at least a portion of an insert therewithin. The slot 604 may be of a height and width which interact with the inserts 402 of the bridge 20, such as to make a strong coupling, while not detracting from the ability to remove the arm 30 from the bridge 20.

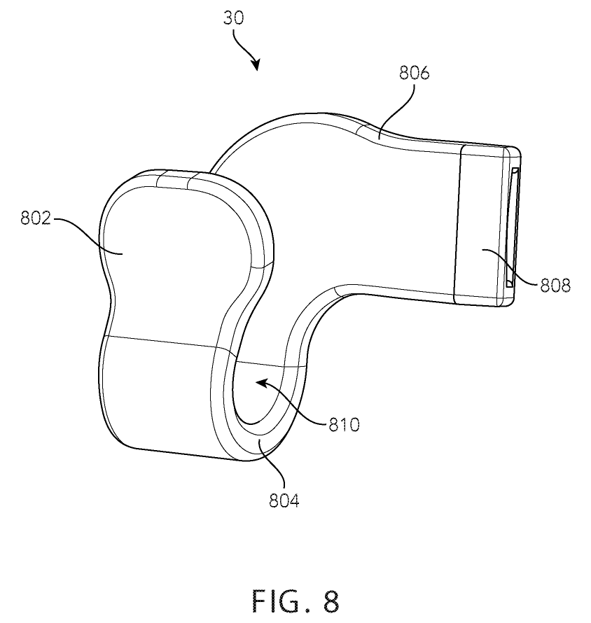

The nasal coupling mechanism 602 may be sized and/or configured with respect to the width of an ala of a user’s nose, as well as the depth of their nostril. In embodiments including a kit, multiple arms 30 may be included, the arms 30 including different combinations of height (e.g., the height 608 of an inner portion of the arm), which is inserted into the nostril, as well as the width of the opening (e.g., the space between inner portion 802 and outer portion 806 as shown and described in FIG. 8 below) for interacting with the user’s alae. In this way, the user may select an arm 30 based on personal preference and/or comfort involving how far the arm 30 sits within their nose while in use, as well as how tightly the arm 30 presses against the user’s alae while in use. Additionally, magnets may be provided within arms 30 to cause the nasal coupling mechanism 602 to hold more strongly to the user’s alae. In additional or alternative embodiments, a portion of the arm 30 may include a magnetized material (e.g., steel, etc.) for interacting with a magnet in a different portion of the arm 30 to cause the nasal coupling mechanism 602 to hold more strongly to the user’s alae.

The arm includes four main portions – an “outer” portion, which, when in use, is disposed around or about the outside of the user’s nose (dorsum), and includes the slot 604 for coupling the arm with the bridge (or is otherwise attached to a “coupling” portion), an “inner” portion, which, when in use, is disposed within the user’s nose, and an “ala contacting” portion, which connects the outer portion to the inner portion, and, when in use, may at least partially rest upon the underside of the user’s ala. FIG. 8 illustrates this in greater depth.

The length of the inner portion may be chosen, based on a selected arm 30, in order to provide an insertion depth that is comfortable for the user. The ala contacting portion may be chosen, based on a selected arm 30, in order to provide a width between the inner portion and the outer portion that fits about/is comfortable when placed about a user’s ala. This may be determined by the user, as well as the width of the user’s ala. For example dimensions of the arm 30, refer to FIGS. 6E and 6F below. In embodiments including magnets, the magnets may be disposed in the inner portion and/or the outer portion such that the inner portion magnet is attracted to the outer portion magnet about the tissue of the nose above the ala, thereby securing the arm to the user’s nose.

The arms 30 may be formed from silicon, plastic, a combination of silicon and plastic, or polymer. In embodiments including a kit, multiple arms 30 may be included, the arms 30 being made from different materials from one another. In this way, the user may select an arm 30 based on a preferred material, such as to avoid an allergy, or discomfort caused by some materials to the specific user.

FIG. 6E illustrates a diagrammatic front view of the arm 30 of FIG. 6A, and FIG. 6F illustrates a diagrammatic side view thereof. A depth 606 of the arm 30 and a height 608 of an inner portion of the arm 30 (e.g., inner portion 802 as shown and described in FIG. 8 below) are shown in FIG. 6E, and a width 610 of an ala contacting portion of the arm 30 (e.g., ala contacting portion 804 as shown and described in FIG. 8 below) are illustrated in FIG. 6F.

The depth 606 of the arm 30 may be from 9.008 mm to 13.524 mm. The height 608 of the inner portion of the arm 30 may be from 12.983 mm to 20.592 mm. The width 610 of the ala contacting portion of the arm 30 may be from 6.75 mm to 9.72 mm. As disclosed above, different arms 30 may be present in a kit, and the different arms 30 may each have different dimensions with respect to the depth 606 of the arm 30, the height 608 of the inner portion of the arm 30, and the width 610 of the ala contacting portion of the arm 30.

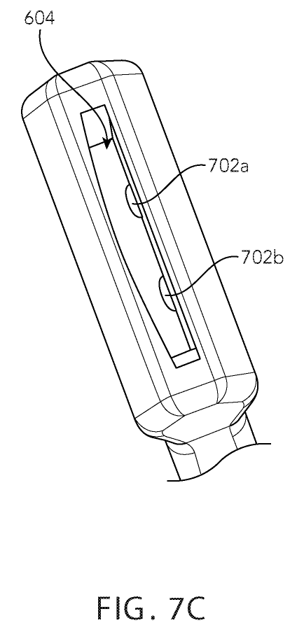

FIGS. 7A-7C illustrate partial views of a slot 604 of an arm 30, according to some embodiments. As seen within the slot 604, male coupling mechanisms 702 may be present and disposed about an inner surface of the slot 604. As shown in FIG. 7A, two male coupling mechanisms 702a and 702b may be disposed within the slot 604 on opposite walls of the slot 604. As shown in FIGS. 7B and 7C, the two male coupling mechanisms 702a and 702b may be disposed along the same wall within the slot 604.

In alternate embodiments, one male coupling mechanism 702 is disposed within the slot 604. In further embodiments, a greater number than two male coupling mechanisms 702 are disposed within the slot 604. In still further embodiments, the number of male coupling mechanisms 702 within the slot 604 of the first arm 30a does not match the number of male coupling mechanisms 702 within the slot 604 of the second arm 30b. It may be desirable to align the number and positioning of male coupling mechanisms 702 with the number and positioning of the associated female coupling mechanisms 502.

The male coupling mechanisms 702 may be protrusions along a wall within the slot 604. In some embodiments, the male coupling mechanisms 702 match, or approximately match, the shape of the female coupling mechanisms 502. The male coupling mechanisms 702 may be configured to interact with the female coupling mechanisms 502 when the inserts 402 are coupled with the slot, so as to help at least partially maintain a coupling between the arm and the bridge. The male coupling mechanisms 702 may be configured to at least partially enter the female coupling mechanisms 502. According to some embodiments, some male coupling mechanisms 702 may reside on a wall of the slot opposite other male coupling mechanisms 702, as illustrated in FIG. 7A.

FIG. 8 illustrates a side view of an arm 30, according to some embodiments. FIG. 8 is presented to break down the specific portions of the arm 30. For example, an inner portion 802 is included. The inner portion 802 may be the portion of the arm 30 which is inserted into a user’s nostril. The inner portion 802 is shown connected to an outer portion 806 via an ala contacting portion 804. In some embodiments, the ala contacting portion 804 curves about the ala of a user’s nose.

According to some embodiments, the outer portion 806 makes contact with an exterior of the user’s nose (dorsum). The outer portion 806 terminates in a coupling portion 808. The coupling portion 808 may be used to couple the arm 30 to the bridge 20, such as by way of the female coupling mechanisms as shown and described in FIGS. 7A-7C above.

Additionally, an opening 810 is illustrated between inner portion 802 and outer portion 806, about which the ala contacting portion 804 extends. The opening 810 may increase in width based on a force placed on either of the inner portion 802 and/or the outer portion 806 to permit a user to extend the arm 30 about their alae. An inherent spring constant of the arm 30 material may permit the arm to snugly fit upon the user’s alae.

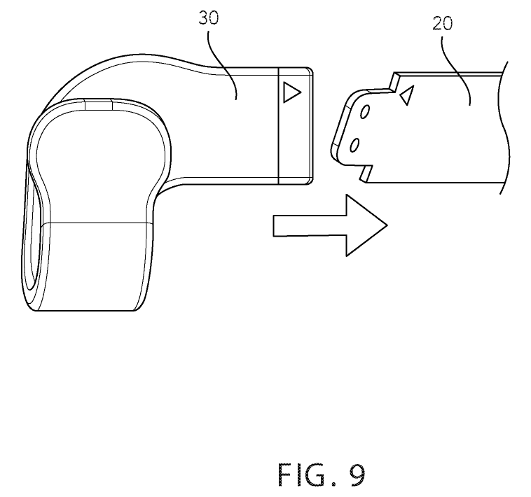

FIG. 9 illustrates a diagrammatic back view of a nasal dilating device 10 being put together, according to some embodiments. As shown, the arm 30 may be placed onto the bridge 20, or, stated another way, a portion of the bridge 20 may be inserted into the arm 30 by way of the female coupling mechanisms and male coupling mechanisms as shown and described above.

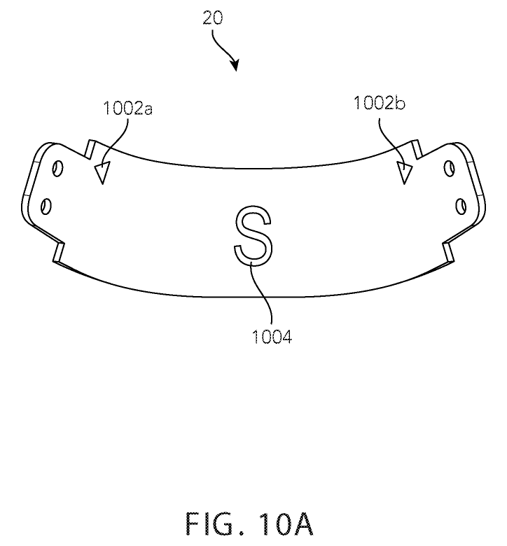

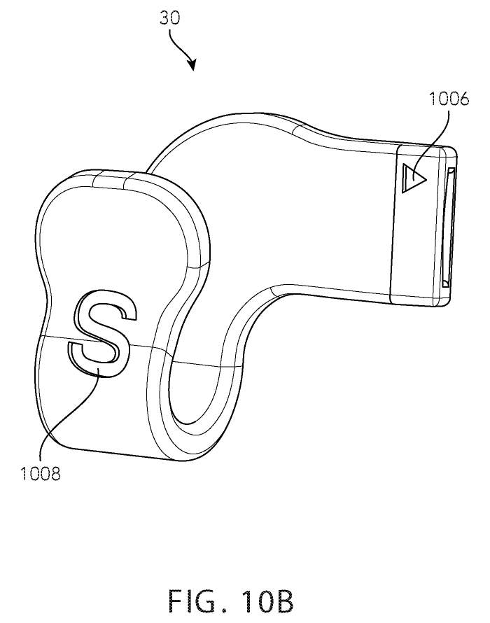

FIG. 10A illustrates a back view of a bridge 20, according to some embodiments. As seen in FIG. 10A, bridge position indicators 1002a and 1002b are present. Bridge position indicators 1002a, 1002b may be used to indicate to a user how to orient the bridge 20 in order to couple the bridge 20 to the arms 30. The bridge position indicators 1002a, 1002b may be positioned and oriented such that they align with an arm position indicator (e.g., arm position indicator 1006 as shown and described in FIG. 10B below) when correctly oriented. The bridge position indicators 1002a, 1002b may be location-specific based on the left and right arms 30. The bridge position indicators 1002a, 1002b are illustrated as triangles in FIG. 10A. In additional or alternate embodiments, any shape may be used. However, it may be preferable to use a shape that has a corner that more obviously points in a direction, such as a triangle, a diamond, etc., in order to better orient the bridge 30 with the arms 30.

Also shown in FIG. 10A, a bridge size indicator 1004 appears as a letter “S” on a back surface of the bridge 20. The bridge size indicator 1004 may indicate to a user what size the bridge 20 is. An “S” is used here to indicate “small.” In this theme, indicators such as “2XS,” “XXS,” “XS,” “M,” “L,” “XL,” “XXL,” “2XL,” and other variants may also be used to indicate various different sizes for a respective bridge. In additional or alternate embodiments, the bridge size indicator 1004 may be a numeric indicator such as the numbers one through five, with one being the smallest size and five being the largest size, or vice versa. The numeric indicators could be spelled out, or shown as Arabic numerals. In additional or alternate embodiments, the bridge size indicator 1004 may be a shape, such as a circle, square, triangle, etc., where the relational size of the shape between different bridges 20 indicates the relational size of the bridges 20 themselves.

FIG. 10B illustrates a side view of an arm 30, according to some embodiments. As seen in FIG. 10B, an arm position indicator 1006 is present. Arm position indicator 1006 may be used to indicate to a user how to orient the arm 30 in order to couple the arm 30 to the bridge 20. The arm position indicator 1006 may be positioned and oriented such that it aligns with the bridge position indicator 1002a or 1002b when correctly oriented. The arm position indicator 1006 is illustrated as a triangle in FIG. 10B. In additional or alternate embodiments, any shape may be used. However, it may be preferable to use a shape that has a corner that more obviously points in a direction, such as a triangle, a diamond, etc., in order to better orient the arm 30 with the bridge 20.

Also shown in FIG. 10B, an arm size indicator 1008 appears as a letter “S” on the inner portion of the arm 30 (e.g., inner portion 802 in FIG. 8 above). The arm size indicator 1008 may indicate to a user what size the arm 30 is. An “S” is used here to indicate “small.” In this theme, indicators such as “2XS,” “XXS,” “XS,” “M,” “L,” “XL,” “XXL,” “2XL,” and other variants may also be used to indicate various different size for a respective arm. In additional or alternate embodiments, the arm size indicator 1008 may be a numeric indicator such as the numbers one through five, with one being the smallest size and five being the largest size, or vice versa. The numeric indicators could be spelled out, or shown as Arabic numerals. In additional or alternate embodiments, the arm size indicator 1008 may be a shape, such as a circle, square, triangle, etc., where the relational size of the shape between different arms 30 indicates the relational size of the arms 30 themselves.

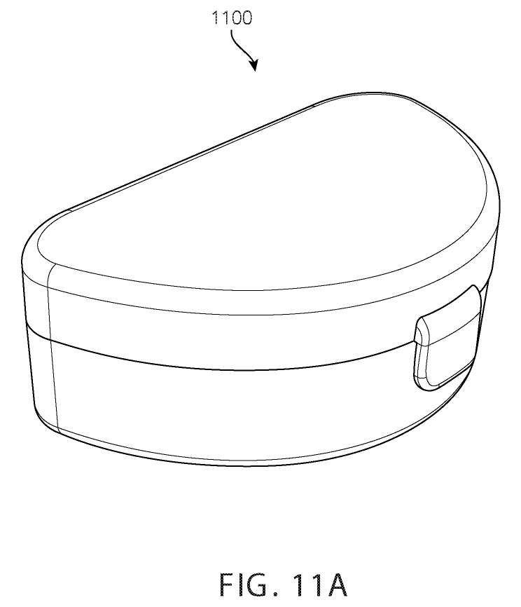

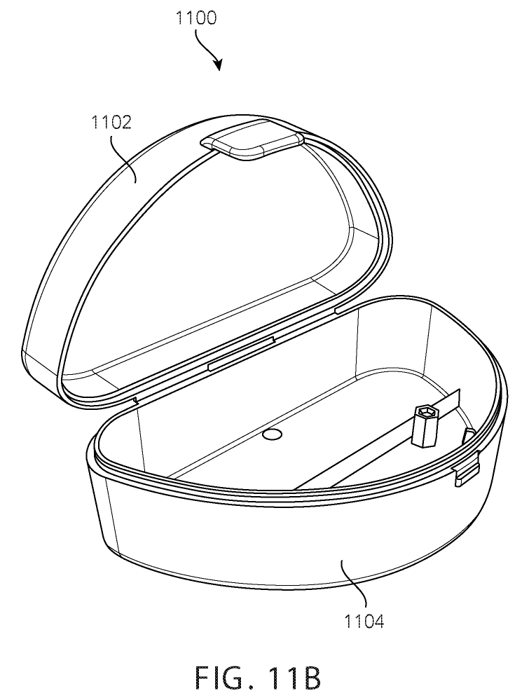

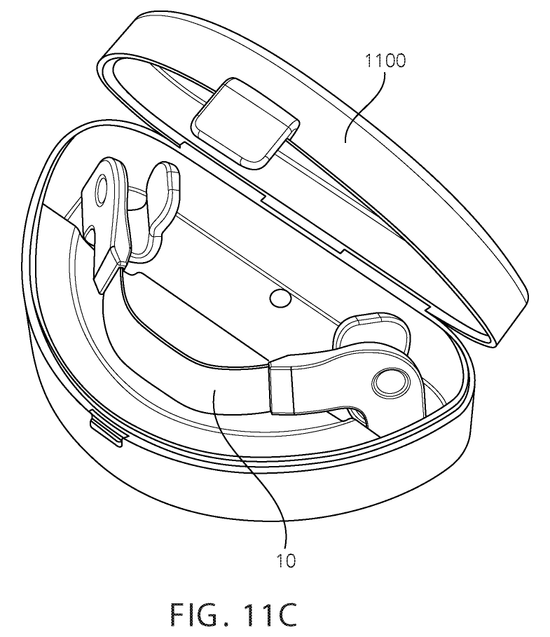

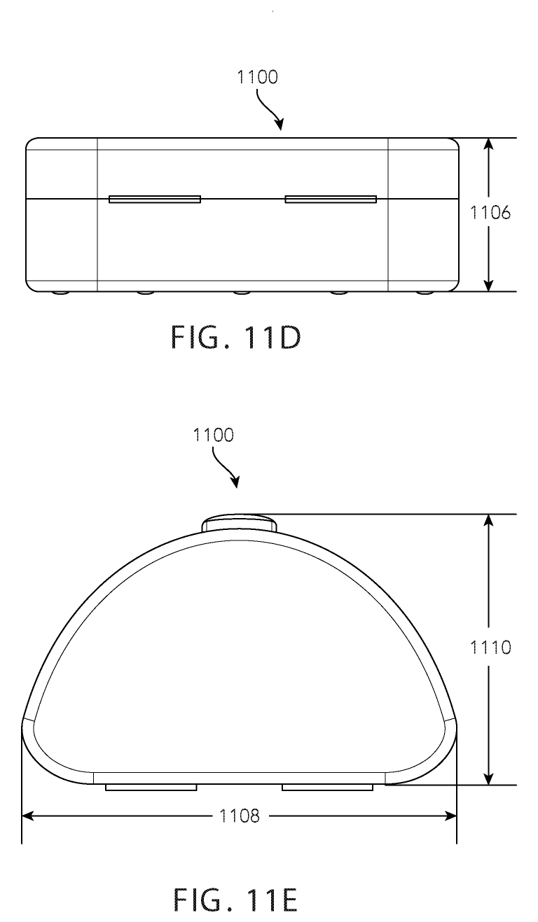

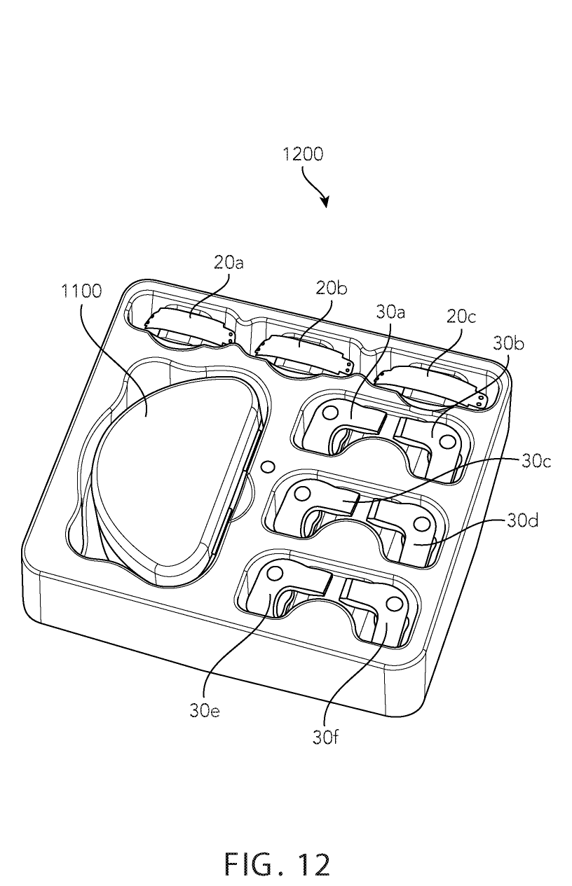

FIG. 11A illustrates a perspective view of a case 1100, according to some embodiments. In embodiments including a kit, a case 1100 may be provided. The case 1100 may be a carrying case. The case may be sized and configured to hold multiple bridges 20 and arms 30, in any combination as described above, to facilitate a user in storing their devices and finding specific components for customizing their devices. The case 1100 may be made from plastic (such as transparent plastic, translucent plastic, opaque plastic, colored plastic, etc.), plastic and foam, faux fur, nylon, metal, or combinations thereof.

FIG. 11B illustrates a perspective view of the case 1100 of FIG. 11A in an open configuration, according to some embodiments. In some embodiments, the case 1100 includes a hinge such that the case 1100 can be opened without individual pieces separating from one another. When opened, the case 1100 can be distinguished as a lid 1102 and a container 1104. In additional or alternative embodiments, the case 1100 may not include a hinge, and instead include a separate lid 1102 and container 1104. In such embodiments, the lid 1102 and the container 1104 may couple to one another when in a closed configuration through a snap fit, a friction fit, etc.

FIG. 11C illustrates a perspective view of the case 1100 of FIG. 11A in an open configuration with a nasal dilating device 10 inside, according to some embodiments. The embodiment illustrated in FIG. 11C shows a case 1100 sized and configured to accept a single nasal dilating device 10 in order to minimize the footprint of the case 1100. In additional or alternative embodiments, the case 1100 may be larger so as to permit the carriage of a greater number of nasal dilating devices 10, to permit size customization on the go.

FIG. 11D illustrates a diagrammatic front view of the case 1100 of FIG. 11A, and FIG. 11E illustrates a diagrammatic top view thereof. A height 1106 of the case 1100 is shown in FIG. 11D, and a length 1108 of the case 1100 and a width 1110 of the case 1100 are shown in FIG. 11E.

The height 1106 of the case 1100 may be from 20.758 mm to 29.892 mm. The length 1108 of the case 1100 may be from 59.608 mm to 85.836 mm. The width 1110 of the case 1100 may be from 34.675 mm to 49.932 mm.

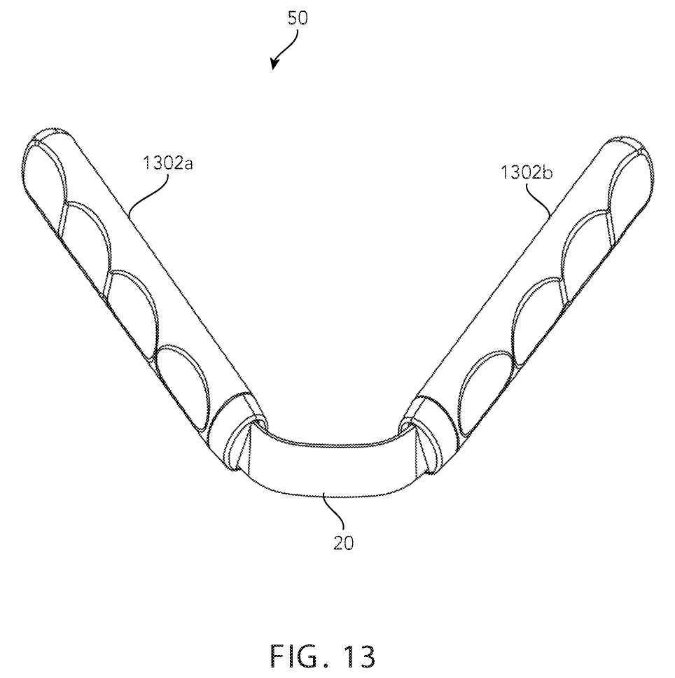

FIG. 12 illustrates a packing tray 1200, according to some embodiments. As shown in FIG. 12, the packing tray 1200 may include cavities for receiving the multiple parts of a kit. Specifically illustrated are a case 1100, bridges 20a, 20b, and 20c, and arms 30a, 30b, 30c, 30d, 30e, and 30f. In additional or alternate embodiments, the packing tray may be larger, so as to hold additional cases 1100, bridges 20, and/or arms 30, or the packing tray may be smaller, so as to hold fewer bridges 20 and/or arms 30, or to exclude the case 1100.

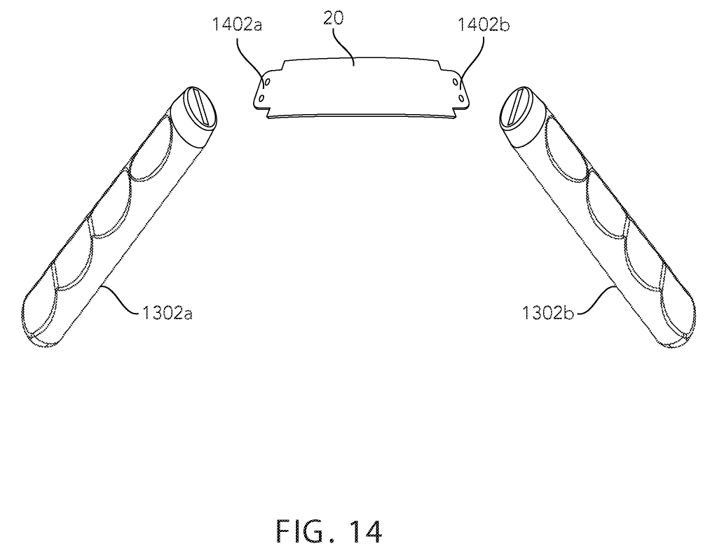

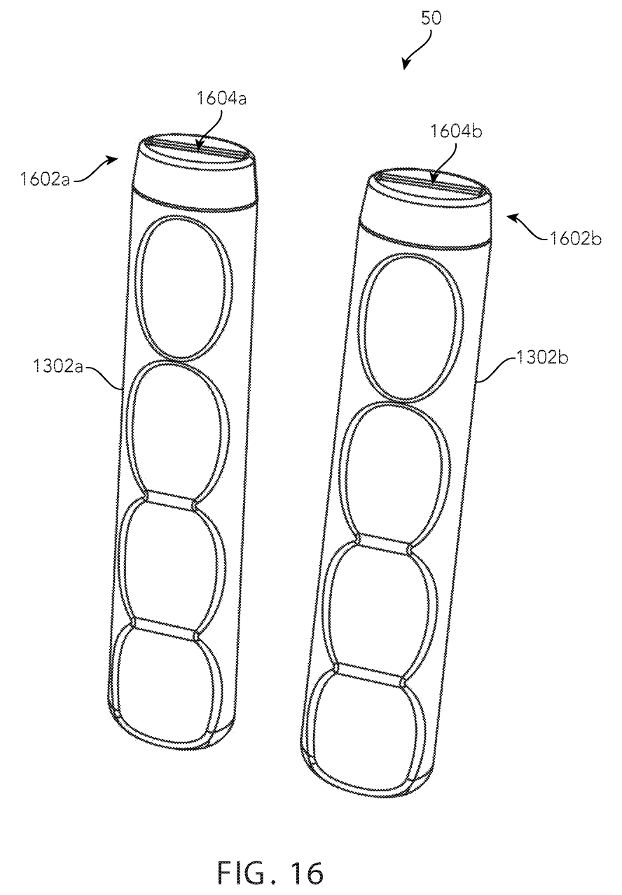

FIG. 13 shows a perspective view of a device, or tool, 50. In some embodiments, the tool 50 includes a first handle portion 1302a and a second handle portion 1302b. The first handle portion 1302a and the second handle portion 1302b may be configured to couple to the bridge 20, as demonstrated in FIG. 13. In some embodiments, the first handle portion 1302a and the second handle portion 1302b are used to alter the radius of curvature of the bridge 20, as will be discussed in greater detail with reference to FIGS. 15A and 15B. The first handle portion 1302a and the second handle portion 1302b may be configured to detachably couple to the bridge 20, such that the bridge 20 can be coupled to the tool 50, adjusted, decoupled from the tool 50, and coupled to the arms 30 for use in nasal dilation, as previously discussed in this disclosure.

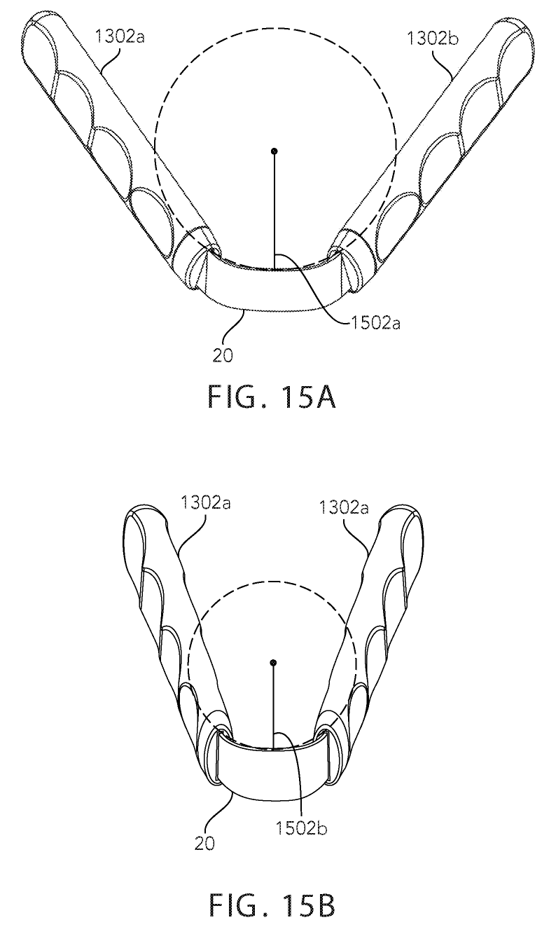

FIG. 14 illustrates the bridge 20 decoupled from the first handle portion 1302a and the second handle portion 1302b. In some embodiments, the bridge 20 includes a first end 1402a and a second end 1402b, located opposite the first end 1402a. As previously discussed in this disclosure, the bridge 20 may include a first insert 402a and a second insert 402b. In some embodiments, the first insert 402a is disposed along the first end 1402a, and the second insert 402b is disposed along the second end 1402b.

When the bridge 20 couples to the tool 50, the first handle portion 1302a may be configured to receive at least a portion of the first end 1402a of the bridge 20, and the second handle portion 1302b may be configured to receive at least a portion of the second end 1402b of the bridge 20. In some embodiments, the first handle portion 1302a is configured to receive the first insert 402a, and the second handle portion 1302b is configured to receive the second insert 402b. The first handle portion 1302a may be configured to receive the second insert 402b, and the second handle portion 1302b may be configured to receive the first insert 402a.

Coupling the bridge 20 to the first handle portion 1302a and the second handle portion 1302b may enable the user 40 to adjust the bridge 20, as illustrated in FIGS. 15A and 15B. In some embodiments, the bridge 20 is manufactured with a predetermined radius of curvature, or angle of travel, as discussed earlier in this disclosure. This radius of curvature, combined with the inherent spring constant of the bridge 20, will attempt to bring the bridge 20 back to its original position, outside of external force. When assembled and coupled to the user 40, this tension in the bridge 20 will cause the arms 30 to separate the alae, along with further portions of the nostril, away from the septum, thereby opening the nasal cavity (as described herein). This, in turn, may facilitate the opening of the nasal airway, easing air passage through this nasal airway.

As discussed with reference to FIG. 10A, the bridge 20 may be manufactured in a number of different sizes to enable the user 40 to select the best fit for optimal nasal airway opening. However, even with the ability to select from different sized bridges 20, the user 40 may desire to fine-tune the fit of the bridge 20 by altering the radius of curvature. For example, FIG. 15A may be understood as showing a bridge 20 in its standard form and shape after manufacturing. The radius of curvature 1502a shown in FIG. 15A may be ideal for a user 40 with a wide and/or flat nose to achieve a comfortable and effective fit of the nasal dilating device 10. Comparatively, FIG. 15B illustrates a bridge 20 that has been adjusted to have a smaller radius of curvature 1502b, which may provide a more comfortable and effective fit for a user 40 with a narrow nose.

In some embodiments, the tool 50 provides a mechanism for the user 40 to customize the fit of the bridge 20. By using the first handle portion 1302a and/or the second handle portion 1302b, the user 40 may be able to manipulate the shape of the bridge 20, namely by altering the radius of curvature. It should be noted that though FIG. 15B demonstrates a narrower bridge 20, the tool 50 may also be used to widen the bridge 20 (i.e., increase the radius of curvature) beyond what is illustrated in FIG. 15A. It may also be possible to decrease the radius of curvature beyond what is illustrated in FIG. 15B.

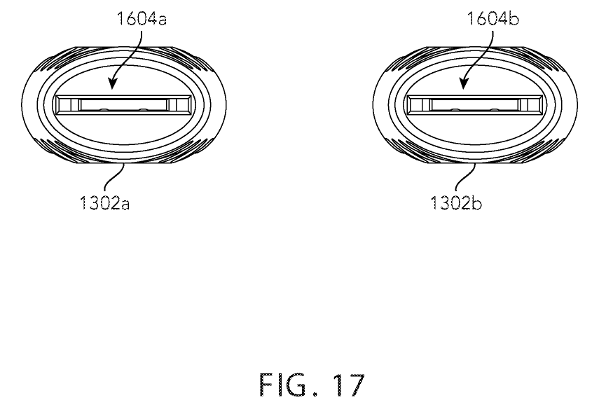

FIG. 16 shows a perspective view of the tool 50. In some embodiments, the first handle portion 1302a includes a first end 1602a, and the second handle portion 1302b includes a first end 1602b. The tool 50 may include a first slot 1604a and a second slot 1604b. In some embodiments, the first slot 1604a is disposed along the first end 1602a of the first handle portion 1302a, as illustrated in FIG. 16. Similarly, the second slot 1604b may be disposed along the first end 1602b of the second handle portion 1302b. According to some embodiments, the first slot 1604a and the second slot 1604b are configured to receive the first insert 402a and the second insert 402b, respectively, of the bridge 20 when the bridge 20 couples to the tool 50.

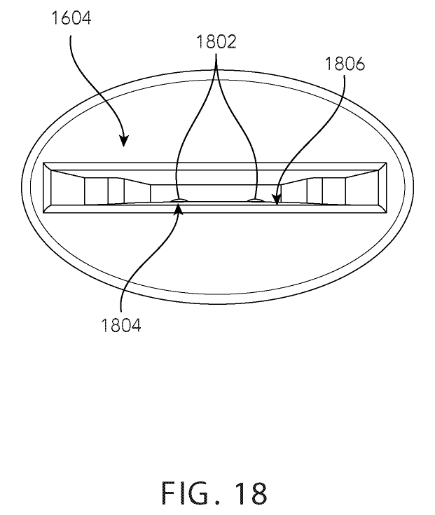

FIG. 17 illustrates a top view of the tool 50, including the first slot 1604a and the second slot 1604b, according to some embodiments. As shown, the first slot 1604a and the second slot 1604b may be located substantially centrally along the respective first end of the respective handle portion 1302a, 1302b. Alternatively, the first slot 1604a and/or the second slot 1604b may be located off-center along a first end of the first handle portion 1302a and/or the second handle portion 1302b.

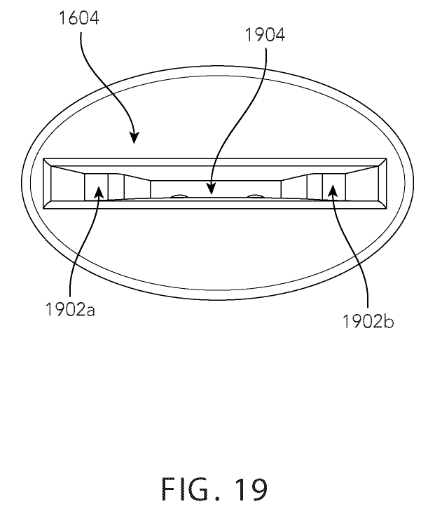

FIG. 18 illustrates a more detailed view of a single slot 1604, which may be thought of as representing either the first slot 1604a or the second slot 1604b. In some embodiments, the first slot 1604a and the second slot 1604b are substantially the same. It should be noted that though FIGS. 18-20 show a single slot 1604, the components discussed with reference to these figures should be understood as being applicable to both the first slot 1604a and the second slot 1604b.

In some embodiments, the slot 1604 includes male coupling mechanisms 1802 configured to detachably couple the first handle portion 1302a and the second handle portion 1302b to the bridge 20, similar to the male coupling mechanisms 702 of the arm 30. The male coupling mechanisms 1802 may include at least one protrusion 1804 located along at least one wall 1806 of the slot 1604. The male coupling mechanisms 1802 may be disposed within the slot 1604, and the at least one protrusion 1804 may be located along any suitable wall 1806 of the slot 1604. In some embodiments, the male coupling mechanisms 1802 includes two protrusions 1804 located along the same wall 1806, as demonstrated in FIG. 18. Alternatively, the protrusions 1804 may be located along opposite walls 1806, similar to as illustrated for the male coupling mechanisms 702 of the arm in FIG. 7A. In some embodiments, the male coupling mechanisms 1802 includes a single protrusion 1804. The male coupling mechanisms 1802 may include more than two protrusions 1804.

In some embodiments, the male coupling mechanisms 1802 match, or approximately match, the shape of the female coupling mechanisms 502 of the bridge 20. The male coupling mechanisms 1802 may be configured to interact with the female coupling mechanisms 502 when the inserts 402 are coupled with the slot 1604, so as to help at least partially maintain a coupling between the tool 50 and the bridge 20. The male coupling mechanisms 1802 may be configured to at least partially enter the female coupling mechanisms 502.

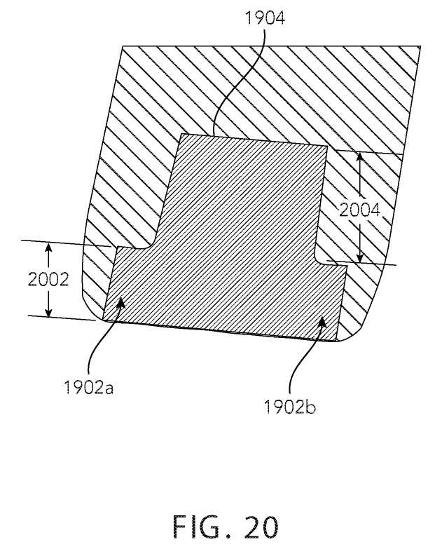

In some embodiments, the slot 1604 includes a first side portion 1902a, a second side portion 1902b, and a middle portion 1904 located between the first side portion 1902a and the second side portion 1902b, as indicated in FIG. 19. The first side portion 1902a and the second side portion 1902b may each define a first depth 2002, as illustrated in FIG. 20, which may be considered an X-ray view of an end 1602 of the handle portion 1302. The middle portion 1904 may define a second depth 2004, as also illustrated in FIG. 20. In some embodiments, the second depth 2004 is greater than the first depth 2002. Accordingly, the varying depths may define a stepped shape or configuration within the slot 1604.

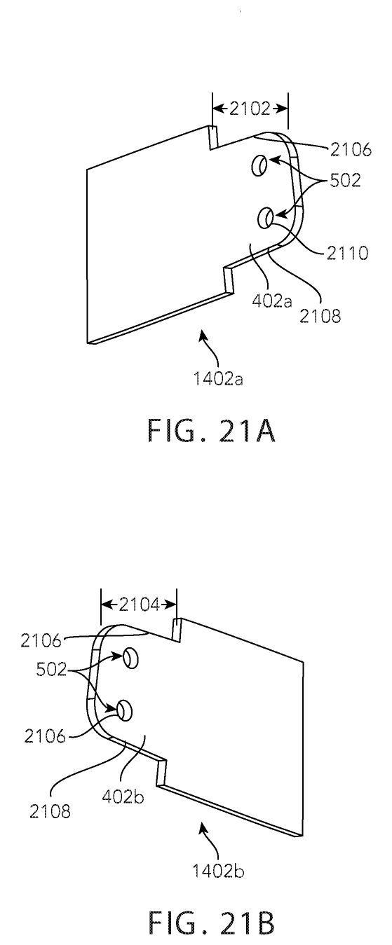

The stepped shape of the slot 1604 may be configured to correspond to the shape of the first end 1402a and the second end 1402b of the bridge 20 to facilitate coupling of the first insert 402a and the second insert 402b within the slot 1604. For example, in some embodiments, the first insert 402a defines a first length 2102 and the second insert 402b defines a second length 2104, as indicated in FIGS. 21A and 21B, respectively. The first length 2102 and the second length 2104 may both correspond to the second depth 2004 such that the first insert 402a and the second insert 402b are configured to be received within the middle portion 1904 of the slot 1604 (e.g., for a respective handle portion 1302a, 1302b).

Further, the bridge 20 may be arranged and configured such that the edges of the main body of the bridge 20, the shoulder-like areas located on either side of the insert 402, are configured to fit within the slot 1604 along the first side portion 1902a and the second side portion 1902b when the bridge 20 is coupled to the first handle portion 1302a and/or the second handle portion 1302b.

It should be noted that, due to the curvature of the bridge 20, the inserts 402 may not be perfectly symmetrical when reflected along a horizontal axis. For example, the top edge 2106 of each insert 402, as shown in FIGS. 21A and 21B, may be shorter than the bottom edge 2108. In some embodiments, the top edge 2106 is longer than the bottom edge 2108. The dimension difference may be as small as only a fraction of a millimeter and, as a result, may not impact the ability of the bridge 20 to securely couple to the first handle portion 1302a and/or the second handle portion 1302b. In addition, in some embodiments, the middle portion 1904 of the slot 1604 is angled slightly, such that one side edge of the middle portion 1904 defines a slightly greater depth than the other side edge. This configuration may correspond to the angled shape of the inserts 402, thereby allowing the inserts 402 to couple precisely within the middle portion 1904.

Also shown in FIGS. 21A and 21B is that, in some embodiments, the female coupling mechanisms 502 of the bridge 20 define at least one aperture 2110. The at least one aperture 2110 may be configured to receive the at least one protrusion 1804 in order to couple the bridge 20 to the first handle portion 1302a and/or the second handle portion 1302b via the female coupling mechanisms 502 and the male coupling mechanism 1802. In some embodiments, rather than a complete aperture 2110, the female coupling mechanisms 502 defines a groove, dent, divot, or recessed portion in the insert 402. The number of female coupling mechanisms 502 may be configured to correspond with, or match, the number of male coupling mechanisms 1802. Alternatively, the number of female coupling mechanisms 502 may be different than the number of male coupling mechanisms 1802. In some embodiments, the female coupling mechanisms 502 are configured to align with the male coupling mechanisms 1802 when the bridge 20 is coupled to the tool 50.

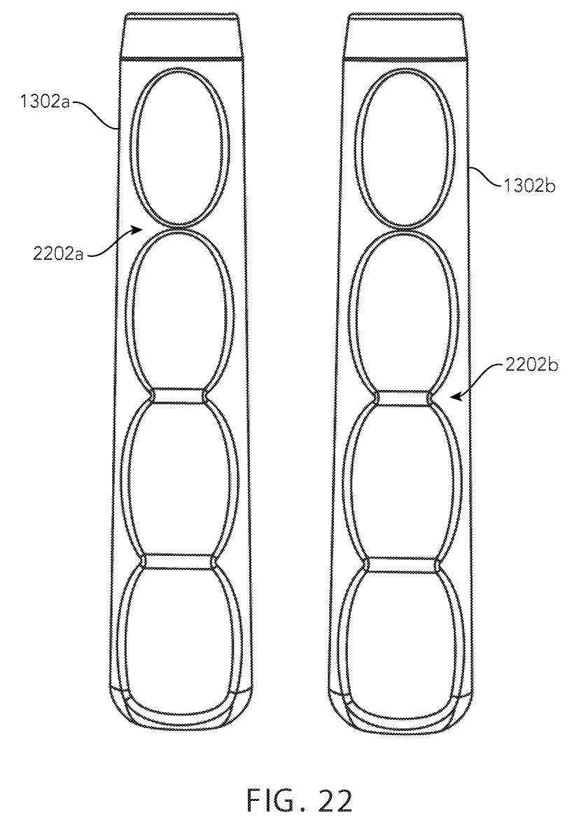

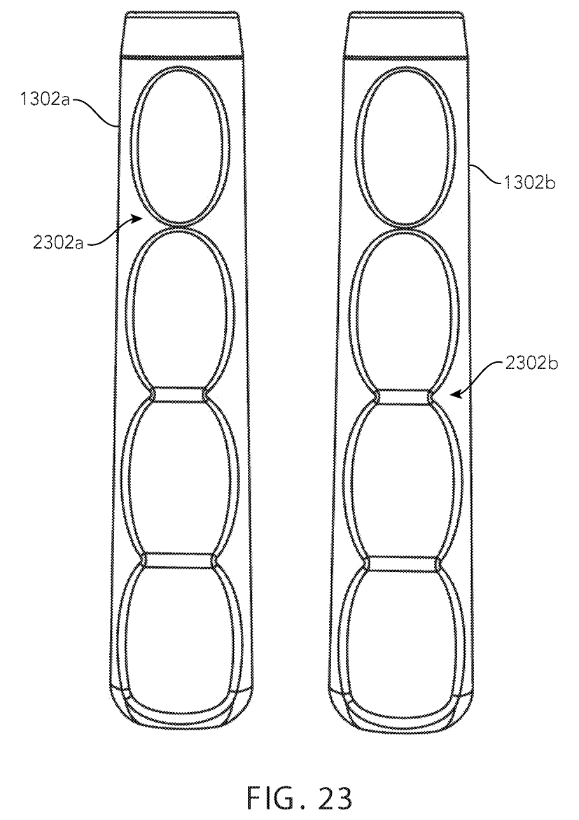

FIGS. 22 and 23 illustrate front and back views, respectively, of the tool 50. In some embodiments, the first handle portion 1302a includes a first face 2202a and a second face 2302a located opposite the first face 2202a. Similarly, the second handle portion 1302b may include a first face 2202b and a second face 2302b located opposite the first face 2202b. In some embodiments, the first face 2202 and the second face 2302 are substantially identical to one another.

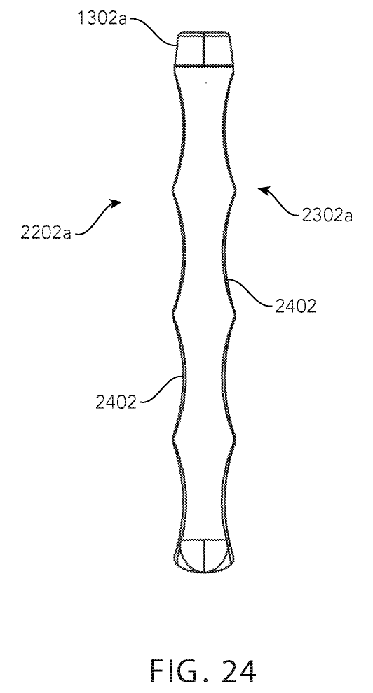

FIG. 24 illustrates a side view of the first handle portion 1302a, wherein the first face 2202 and the second face 2302 may each include at least one grip area 2402. In some embodiments, the at least one grip area 2402 provides an area for the user 40 to hold the first handle portion 1302a and/or the second handle portion 1302b while adjusting the bridge 20. The at least one grip area 2402 may include a series of ridges, a textured area, at least one fingerhold, or any suitable element that provides increase comfort and/or stability to the user 40 when handling the tool 50. It should be noted that though FIG. 24 is labeled as the first handle portion 1302a, it may also be considered as representing the second handle portion 1302b.

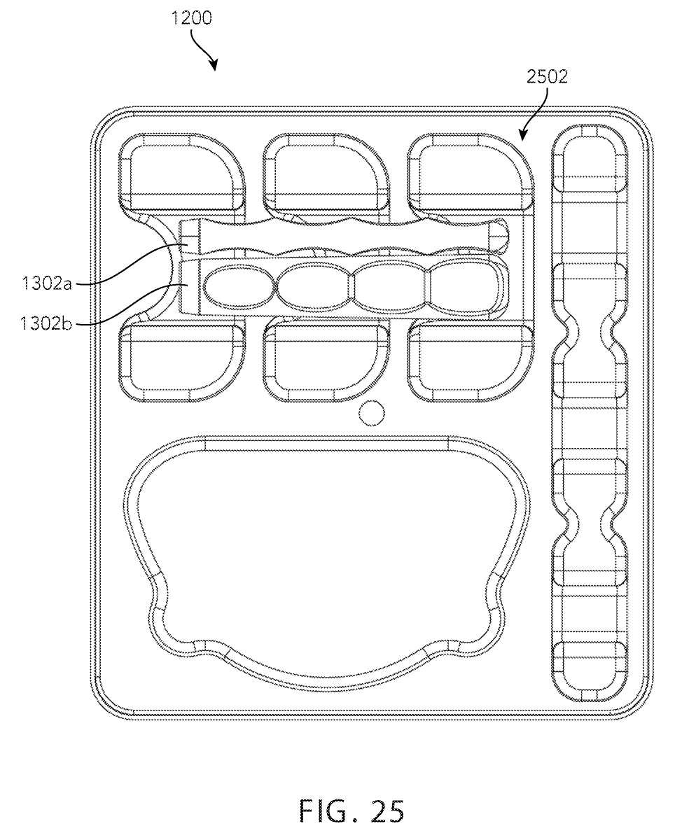

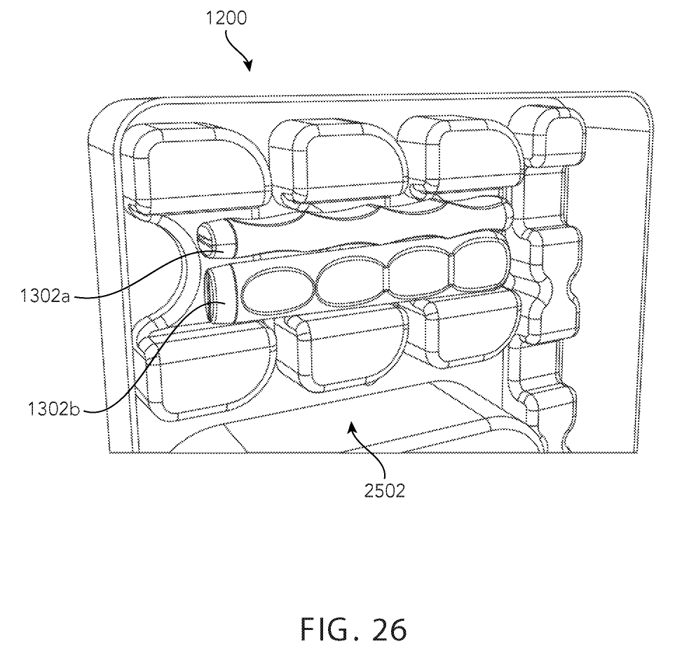

FIGS. 25 and 26 illustrate the packing tray 1200, previously shown in FIG. 12. More specifically, FIGS. 25 and 26 illustrate the back surface 2502 of the packing tray 1200, while FIG. 12 shows the front surface of the packing tray 1200. In some embodiments, as demonstrated in FIGS. 25 and 26, the first handle portion 1302a and the second handle portion 1302b are arranged and configured to be stored along the back surface 2502 of the packing tray 1200. The first handle portion 1302a and the second handle portion 1302b may be stored along the back surface 2502 opposite the plurality of arms 30. Stated differently, the back side of the area corresponding to the area in the front (FIG. 12) that is used to hold the arms 30 may include an indented portion sized to receive the first handle portion 1302a and the second handle portion 1302b. The first handle portion 1302a and the second handle portion 1302b may be stored along any suitable portion of the packing tray 1200, including the front surface or the back surface 2502.

In some embodiments, the disclosure includes a kit 2702, as illustrated in FIG. 27. The kit 2702 may include the tool 50, which in turn may include the first handle portion 1302a and the second handle portion 1302b. In some embodiments, the first handle portion 1302a includes a first slot 1604a with male coupling mechanisms 1802, and the second handle portion 1302b includes a second slot 1604b with male coupling mechanisms 1802.

The kit 2702 may also include a bridge 20, which, in some embodiments, is a first bridge 20a. The first bridge 20a may include a first end 1402a and a second end 1402b. In some embodiments, the first end 1402a includes a first insert 402a and female coupling mechanisms 502, while the second end 1402b includes a second insert 402b and female coupling mechanisms 502. The kit 2702 may include a second bridge 20b and a third bridge 20c. In some embodiments, the first bridge 20a, the second bridge 20b, and the third bridge 20c define different bridge sizes, as previously discussed in this disclosure. For example, the first bridge 20a may define a first bridge size, the second bridge 20b may define a second bridge size, and the third bridge 20c may define a third bridge size.

It should be noted that, like the first bridge 20a, the second bridge 20b and the third bridge 20c may include ends 1402, inserts 402, and female coupling mechanisms 502. Accordingly, the tool 50 may be used to change the radius of curvature of any of the first bridge 20a, the second bridge 20b, and the third bridge 20c. In some embodiments, the kit 2702 includes more than three bridges 20. The kit 2702 may include fewer than three bridges 20. The tool 50 may be configured to adjust any number of bridges 20 included with the kit 2702 (as described herein, e.g., FIGS. 13-26).

In some embodiments, the kit 2702 includes a first set of arms 2704a, including a first arm 30a and a second arm 30b. In some embodiments, the first arm 30a and the second arm 30b each include a slot 604 and male coupling mechanisms 702. The kit 2702 may also include a second set of arms 2704b, including a third arm 30c and a fourth arm 30d, and a third set of arms 2704c, including a fifth arm 30e and a sixth arm 30f. In some embodiments, the first set of arms 2704a, the second set of arms 2704b, and the third set of arms 2704c define different sizes. For example, the first set of arms 2704a may define a first arm size, the second set of arms 2704b may define a second arm size, and the third set of arms 2704c may define a third arm size. The kit 2702 may include more than three sets of arms 2704. In some embodiments, the kit 2702 includes fewer than three sets of arms 2704.

In some embodiments, like the first arm 30a and the second arm 30b, each of the third arm 30c, the fourth arm 30d, the fifth arm 30e, and the sixth arm 30f includes a slot 604 and male coupling mechanisms 702. Accordingly, any of the first arm 30a, the second arm 30b, the third arm 30c, the fourth arm 30d, the fifth arm 30e, and the sixth arm 30f may be able to couple to any of the first bridge 20a, the second bridge 20b, and the third bridge 20c for use to dilate the nasal passageway of the user 40, as described throughout this disclosure. Providing multiple sizes of bridges 20 and arms 30 may enable the user 40 to find the best combination of components to ensure the best fit. For example, one user 40 may find that the first bridge 20a with the second set of arms 2704b is the best fit, while another user 40 may find that the third bridge 20c with the first set of arms 2704a is the best fit for them. The tool 50 may be used to fine-tune the fit of the selected bridge 20, which, combined with several size options of bridges 20 and arms 30, provides a highly customizable fit for each user 40.

As also indicated in FIG. 27, the kit 2702 may include a case 1100, previously shown in FIGS. 11A-11E. The case 1100 may be sized and configured to store at least one of a bridge 20, a set of arms 2704, and the tool 50. The case 1100 may be sized and configured to store multiple bridges 20, multiple sets of arms 2704 and/or the tool 50. In some embodiments, the kit 2702 may be thought of as including all of the elements illustrated in FIGS. 12 and 25. Stated differently, the kit 2702 may include every component stored in the packing tray 1200.

In some embodiments, each of the first handle portion 1302a and the second handle portion 1302b is about 65 millimeters (mm) long. The first handle portion 1302a and the second handle portion 1302b may define a length that ranges from about 32.5 mm to about 97.5 mm. The first handle portion 1302a and the second handle portion 1302b may define a tapered shape from the respective first end 1602a, 1602b to the opposite end, or second end. In some embodiments, the second end is wider than the first end 1602. The second end may define a width of about 13 mm, and the first end 1602 may define a width of about 10 mm. In some embodiments, the width of the second end ranges from about 6.5 mm to about 19.6 mm, and the width of the first end 1602 ranges from about 5 mm to about 15 mm. The depth, or thickness, of the second end may be about 7 mm. In some embodiments, the depth of the second end ranges from about 3.5 mm to about 10.5 mm.

In one embodiment, the first handle portion 1302a and the second handle portion 1302b each define a length of 65 mm, a width at the first end 1602 of 10.11 mm, a width at the second end of 12.99 mm, and a depth/thickness at the second end of 7.31 mm.

In some embodiments, the nasal dilating device is custom-fit for each user 40 based on each user’s 40 nasal dimensions. Accordingly, the device may include a bridge with integrated arms, rather than the removable arms 30 discussed herein. The one-piece device may achieve the same nasal dilating effects as the device 10 disclosed herein.

Some of the components listed herein use the same number from figure to figure. It should be appreciated that these components use the same numbers solely for ease of reference and to facilitate comprehension for the reader. While these components may use the same numbers, differences may be present in these components as illustrated in the various figures in which they appear and as described in the specification herein.

None of the steps described herein is essential or indispensable. Any of the steps can be adjusted or modified. Other or additional steps can be used. Any portion of any of the steps, processes, structures, and/or devices disclosed or illustrated in one embodiment, flowchart, or example in this specification can be combined or used with or instead of any other portion of any of the steps, processes, structures, and/or devices disclosed or illustrated in a different embodiment, flowchart, or example. The embodiments and examples provided herein are not intended to be discrete and separate from each other.

The section headings and subheadings provided herein are nonlimiting. The section headings and subheadings do not represent or limit the full scope of the embodiments described in the sections to which the headings and subheadings pertain. For example, a section titled “Topic 1” may include embodiments that do not pertain to Topic 1 and embodiments described in other sections may apply to and be combined with embodiments described within the “Topic 1” section.

The various features and processes described above may be used independently of one another, or may be combined in various ways. All possible combinations and subcombinations are intended to fall within the scope of this disclosure. In addition, certain method, event, state, or process blocks may be omitted in some implementations. The methods, steps, and processes described herein are also not limited to any particular sequence, and the blocks, steps, or states relating thereto can be performed in other sequences that are appropriate. For example, described tasks or events may be performed in an order other than the order specifically disclosed. Multiple steps may be combined in a single block or state. The example tasks or events may be performed in serial, in parallel, or in some other manner. Tasks or events may be added to or removed from the disclosed example embodiments. The example systems and components described herein may be configured differently than described. For example, elements may be added to, removed from, or rearranged compared to the disclosed example embodiments.

Conditional language used herein, such as, among others, "can," "could," "might," "may," “e.g.,” and the like, unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require at least one of X, at least one of Y, and at least one of Z to each be present.

The term “and/or” means that “and” applies to some embodiments and “or” applies to some embodiments. Thus, A, B, and/or C can be replaced with A, B, and C written in one sentence and A, B, or C written in another sentence. A, B, and/or C means that some embodiments can include A and B, some embodiments can include A and C, some embodiments can include B and C, some embodiments can only include A, some embodiments can include only B, some embodiments can include only C, and some embodiments can include A, B, and C. The term “and/or” is used to avoid unnecessary redundancy.

The term “substantially” refers to less than or equal to +/−1%, +/−2%, +/−3%, +/−4%, +/−5%, +/−6%, +/−7%, +/−8%, +/−9%, +/−10%, +/−11%, +/−12%, +/−14%, or +/−15% variation. As a non-limiting example, substantially parallel represents a range of −1 to 1 degree difference, −5 to 5 degree difference, or −15 degrees to 15 degrees of difference from being parallel, depending on the embodiments.

While certain example embodiments have been described, these embodiments have been presented by way of example only, and are not intended to limit the scope of the inventions disclosed herein. Thus, nothing in the foregoing description is intended to imply that any particular feature, characteristic, step, module, or block is necessary or indispensable. Indeed, the novel methods and systems described herein may be embodied in a variety of other forms; furthermore, various omissions, substitutions, and changes in the form of the methods and systems described herein may be made without departing from the spirit of the inventions disclosed herein.

We claim:

1. A device, comprising:
a first handle portion; and
a second handle portion,
wherein the first handle portion is configured to receive a first end of a bridge configured to be placed about the bridge of a nose of a user, and the second handle portion is configured to receive a second end of the bridge, wherein the second end of the bridge is located opposite the first end of the bridge,
wherein when the bridge is coupled to the first handle portion and the second handle portion, the first handle portion and the second handle portion may be used to alter a radius of curvature of the bridge.

2. The device of claim 1, wherein a first end of the first handle portion comprises a first slot configured to receive the first end of the bridge, and a first end of the second handle portion comprises a second slot configured to receive the second end of the bridge.

3. The device of claim 2, wherein the first end of the bridge comprises a first insert comprising a first female coupling mechanism, and the second end of the bridge comprises a second insert comprising a second female coupling mechanism.

4. The device of claim 3, wherein the first slot is configured to receive the first insert and the second slot is configured to receive the second insert.

5. The device of claim 4, further comprising a first male coupling mechanism disposed within the first slot and a second male coupling mechanism disposed within the second slot,
wherein the first male coupling mechanism is configured to detachably couple with the first female coupling mechanism when the first slot is coupled with the first insert, and
wherein the second male coupling mechanism is configured to detachably couple with the second female coupling mechanism when the second slot is coupled with the second insert.

6. The device of claim 5, wherein the first male coupling mechanism comprises at least one protrusion located along a wall within the first slot, and the first female coupling mechanism comprises at least one aperture configured to at least partially receive the at least one protrusion of the first male coupling mechanism.

7. The device of claim 6, wherein the second male coupling mechanism comprises at least one protrusion located along a wall within the second slot, and the second female coupling mechanism comprises at least one aperture configured to at least partially receive the at least one protrusion of the second male coupling mechanism.

8. The device of claim 3, wherein the first slot comprises:
a first side portion defining a first depth;
a second side portion spaced from the first side portion and defining the first depth; and
a middle portion located between the first side portion and the second side portion, wherein the middle portion defines a second depth that is greater than the first depth.

9. The device of claim 8, wherein the second slot comprises:
a first side portion defining the first depth;
a second side portion spaced from the first side portion and defining the first depth; and
a middle portion located between the first side portion and the second side portion, wherein the middle portion defines the second depth.

10. The device of claim 9, wherein the first insert defines a first length configured to correspond to the second depth of the middle portion of the first slot, wherein the second insert defines a second length configured to correspond to the second depth of the middle portion of the second slot.

11. The device of claim 1, wherein the first handle portion comprises a first face and a second face located opposite the first face of the first handle portion, wherein each of the first face and the second face of the first handle portion comprises at least one grip area, and wherein the second handle portion comprises a first face and a second face located opposite the first face of the second handle portion, wherein each of the first face and the second face of the second handle portion comprises at least one grip area.

12. A kit, comprising:
a tool comprising a first handle portion and a second handle portion; and
a bridge comprising a first end and a second end located opposite the first end, wherein the bridge is configured to be placed about a bridge of a nose of a user,
wherein the first handle portion is configured to receive at least a portion of the first end of the bridge, and the second handle portion is configured to receive at least a portion of the second end of the bridge,
wherein when the bridge is coupled to the first handle portion and the second handle portion, the first handle portion and the second handle portion may be used to alter a radius of curvature of the bridge.

13. The kit of claim 12, wherein the first handle portion comprises a first slot and a first male coupling mechanism disposed within the first slot, and
wherein the second handle portion comprises a second slot and a second male coupling mechanism disposed within the second slot.

14. The kit of claim 13, wherein the first end of the bridge comprises a first insert comprising a first female coupling mechanism, and the second end of the bridge comprises a second insert comprising a second female coupling mechanism,
wherein the first slot is configured to receive the first insert and the second slot is configured to receive the second insert.

15. The kit of claim 14, wherein the first male coupling mechanism is configured to detachably couple with the first female coupling mechanism when the first slot is coupled with the first insert, and
wherein the second male coupling mechanism is configured to detachably couple with the second female coupling mechanism when the second slot is coupled with the second insert.

16. The kit of claim 12, further comprising:
a first arm configured to detachably couple to the first end of the bridge, wherein at least a portion of the first arm is configured to be inserted within a first nostril of the user; and
a second arm configured to detachably couple to the second end of the bridge, wherein at least a portion of the second arm is configured to be inserted within a second nostril of the user,
wherein the bridge comprises a spring constant such that the bridge is configured to independently pull the first arm and the second arm away from one another to thereby open a nasal airway.

17. The kit of claim 16, wherein the first end of the bridge comprises a first insert comprising a first female coupling mechanism,
wherein the second end of the bridge comprises a second insert comprising a second female coupling mechanism,
wherein the first arm comprises a first slot and a first male coupling mechanism disposed within the first slot,
wherein the second arm comprises a second slot and a second male coupling mechanism disposed within the second slot,
wherein the first male coupling mechanism is configured to detachably couple with the first female coupling mechanism when the first slot is coupled with the first insert, and
wherein the second male coupling mechanism is configured to detachably couple with the second female coupling mechanism when the second slot is coupled with the second insert.

18. The kit of claim 16, wherein the bridge comprises a first bridge defining a first bridge size, and the first arm and the second arm comprise a first set of arms defining a first arm size, the kit further comprising:
a second bridge defining a second bridge size that is different from the first bridge size;
a third bridge defining a third bridge size that is different from the first bridge size and the second bridge size;
a second set of arms defining a second arm size that is different from the first arm size, the second set of arms comprising a third arm and a fourth arm; and
a third set of arms defining a third arm size that is different from the first arm size and the second arm size, the third set of arms comprising a fifth arm and a sixth arm.

19. The kit of claim 18, further comprising a case configured to securably store at least one of the tool, the first bridge, the second bridge, the third bridge, the first set of arms, the second set of arms, and the third set of arms.

20. The kit of claim 19, wherein the tool is sized and configured to detachably couple to any of the first bridge, the second bridge, and the third bridge.
